(19) United States
(12) Patent Application Publication
(10) Pub. No.: US 2024/0272180 A1
(43) Pub. Date: Aug. 15, 2024

(54) SYSTEMS AND METHODS FOR IDENTIFYING AND TREATING NEURODEGENERATIVE DISEASE

(71) Applicant: CND Life Sciences, Inc., Scottsdale, AZ (US)

(72) Inventors: Todd Levine, Paradise Valley, AZ (US); Christopher Gibbons, Brookline, MA (US); Roy Freeman, Brookline, MA (US)

(73) Assignee: CND Life Sciences, Inc., Scottsdale, AZ (US)

(21) Appl. No.: 18/417,920

(22) Filed: Jan. 19, 2024

Related U.S. Application Data
(63) Continuation-in-part of application No. 17/222,970, filed on Apr. 5, 2021.
(60) Provisional application No. 63/005,313, filed on Apr. 4, 2020.

(51) Int. Cl. G01N 33/68; A61B 5/00; G16H 10/40; G16H 50/20

(52) U.S. Cl. CPC G01N 33/6896; A61B 5/4082; G16H 10/40; G16H 50/20; G01N 2440/14; G01N 2800/2835

(57) ABSTRACT

Various embodiments provide a synuclein signature for distinguishing multiple system atrophy (MSA) from Parkinson's disease (PD) in a patient, the synuclein signature comprises a quantitation of total P-SYN across a posterior cervical biopsy site, a distal thigh biopsy site, and a distal leg biopsy site, and a distribution coefficient of P-SYN across the posterior cervical biopsy site, the distal thigh biopsy site, and the distal leg biopsy site.

### CROSS-REFERENCE APPLICATION

This US patent application is a continuation in part patent application of pending U.S. patent application Ser. No. 17/222,970, entitled Systems and Methods for Identifying and Treating a Neurodegenerative Disease, filed Apr. 5, 2021, which claims priority to and the benefit of U.S. Provisional Patent Application Ser. No. 63/005,313, entitled Systems and Methods for Identifying and Treating a Neurodegenerative Disease, filed Apr. 4, 2020, which both are incorporated by reference herein.

### BACKGROUND

Finding an easily accessible and reliable clinical tool to diagnose the diseases collectively defined as ‘synucleinopathies’ is an urgent unmet priority. Synucleinopathies include four overlapping disorders: Parkinson disease (PD), Dementia with Lewy bodies (DLB), multiple system atrophy (MSA) and pure autonomic failure (PAF). All synucleinopathies are characterized by the deposition of phosphorylated α-synuclein in the central and/or peripheral nervous systems resulting in progressive neurological degeneration. There are over 2.5 million people in the United States who have a diagnosis of a synucleinopathy, and an approximately 180,000 are diagnosed every year. Unfortunately, thousands of individuals are undetected or misdiagnosed because there is no simple clinical method to diagnose a synucleinopathy.

Since the diagnosis of a synucleinopathy is based on clinical criteria, the clinical diagnostic accuracy is less than 80%, among movement disorder specialists. MSA is a rapidly progressive neurodegenerative disorder characterized by the presence of autonomic dysfunction and motor features such as Parkinsonism and/or cerebellar ataxia. Autopsy studies of patients with MSA and PD show a high misdiagnosis rate, particularly early in the disease course. Recent autopsy studies in MSA reported a diagnostic accuracy of 62% and 79%. A similar diagnostic accuracy has been reported in autopsy studies of PD; a summary of three autopsy studies of patients with early PD suggest a diagnostic accuracy of only 58%.

This diagnostic inaccuracy has important implications for patient care, patient life planning and therapeutic trials. The response to pharmacotherapy, need for ancillary support, quality of life and rate of progression differs markedly between these disorders. These factors, when coupled with diagnostic inaccuracy, have implications for patient care, therapeutic interventions including clinical trials, prognostication and planning for patients and their families.

An unmet need exists for an accurate diagnostic biomarker for the alpha-synucleinopathies, which identifies alpha-synuclein in tissues and organs outside the central nervous system. There is a need for improved diagnostic accuracy to distinguish MSA from PD.

### SUMMARY

Various embodiments provide systems and methods for identifying the deposition of alpha-synuclein in a skin biopsy from a patient and can include determining if the patient has MSA. The methods and systems can include assessing a patient's deposition profile, as compared to a pathological synuclein signature of phosphorylated alpha-synuclein distribution for MSA; and calculating the probability the patient has MSA.

Various embodiments provide a synuclein signature comprising a cutaneous alpha-synuclein distribution pattern, an assessment of intraepidermal sensory nerve fiber density and a dermal autonomic nerve fiber density. The synuclein signature combined with select clinical features determines a specific diagnostic of distinguishing MSA from PD.

Various embodiments provide systems and methods for combining a patient's clinical features with a patient's deposition profile; and calculating the probability the patient has MSA. The methods and systems can include accessing a data set consisting of a patient's clinical features, a patient's deposition profile, and a pathological synuclein signature of phosphorylated alpha-synuclein distribution; transforming a portion of the data set using a multi-faceted algorithm tool into a result that distinguishes a patient's diagnosis of MSA over PD.

Various embodiments provide systems and methods for combining a patient's pathological features into a synuclein signature profile; and calculating the probability the patient has MSA. The synuclein signature profile can include determining the amount of phosphorylated alpha-synuclein in at least one skin biopsy, calculating the distribution of phosphorylated alpha-synuclein synuclein in at least one skin biopsy, and transforming the synuclein signature profile using a multi-faceted algorithm into a result that distinguishes a patient's diagnosis of MSA over PD. In some embodiments, the synuclein signature profile and patient clinic data can be entered into the multi-faceted algorithm, which transforms the profile and data result that distinguishes a patient's diagnosis of MSA or PD, with a probability of greater than 95%.

Various embodiments provide a system for determining a treatment of a synucleinopathy disorder for a patient. The system can comprise: a multifaceted algorithm tool configured to: calculate a patient synuclein signature comprising a cutaneous alpha-synuclein distribution pattern, an assessment of intraepidermal sensory nerve fiber density and a dermal autonomic nerve fiber density; combine the patient synuclein signature with selected clinical information acquired from the patient; transform the combined patient synuclein signature and selected clinical information with a library of standard synuclein signatures and clinical information; and determine a patient's diagnosis of either MSA or PD, with a probability of greater than 95%.

### DRAWINGS

The patent or application file contains at least one drawing executed in color. Copies of this patent or patent application publication with color drawing(s) will be provided by the Office upon request and payment of the necessary fee.

The present disclosure will become more fully understood from the description and the accompanying drawings, wherein:

FIG. 1 is a flowchart illustrating an exemplary method for determining if a patient has a neurodegenerative disorder from a skin biopsy sample, in accordance with various embodiments;

FIG. 2 is a flowchart illustrating an exemplary method for determining if a patient has a neurodegenerative disorder from a skin biopsy sample with quantitative measurement of protein deposition and inclusion of a multifaceted algorithm to improve sensitivity, specificity, and define disease severity, in accordance with various embodiments;

FIG. 3 is a flow chart illustrating an exemplary method for determining if a patient has a neurodegenerative disorder from a skin biopsy sample defining the quantitative measurement of protein deposition and inclusion of a multifaceted algorithm to differentiate between disease types, in accordance with various embodiments;

FIG. 4 is a flow chart illustrating an exemplary method for determining if a patient has one or more of many neurodegenerative disorder from a skin biopsy sample defining the quantitative measurement of protein deposition and inclusion of a multifaceted algorithm to differentiate between disease types, in accordance with various embodiments;

FIG. 5 is a flowchart illustrating an exemplary multi-facet algorithm tool, in accordance with various embodiments;

FIG. 6 is continuation of a flowchart of FIG. 5 illustrating an exemplary multi-facet algorithm tool, in accordance with various embodiments;

FIG. 7 is an example of the P-SYN deposition within nerve fibers illustrating how this method confirms a diagnosis of synucleinopathy, in accordance with various embodiments;

FIG. 8 is the data set illustrating the quantitation of P-SYN in individuals with different synucleinopathies in order to differentiate between disease subtypes, in accordance with various embodiments;

FIG. 9 is graphical representation of exemplary results of a validation study on 110 patients, in accordance with various embodiments;

FIG. 10 is a cartoon illustrating the distribution of phosphorylated alpha-synuclein in biopsy locations for a patient with MSA and in biopsy locations for a patient with PD, in accordance with various embodiments;

FIG. 11 is a topographical distribution and quantity of P-SYN deposition by biopsy site for each individual subject in an exemplary study, in accordance with various embodiments;

FIG. 12 is a scatter plot of distribution coefficient and P-SYN distribution illustrating various test characteristics that distinguish MSA from PD in an exemplary study, in accordance with various embodiments; and

FIG. 13 is a scatter plot of nerve density and P-SYN distribution illustrating various test characteristics that distinguish MSA from PD in an exemplary study, in accordance with various embodiments.

The drawings described herein are for illustrative purposes only of selected embodiments and not all possible implementations and are not intended to limit the scope of any of the exemplary embodiments disclosed herein or any equivalents thereof. It is understood that the drawings are not drawn to scale. For purposes of clarity, the same reference numbers will be used in the drawings to identify similar elements.

### DESCRIPTION

The following disclosure is merely exemplary in nature and is in no way intended to limit the described embodiments, their application, or uses. The present invention has been described with reference to various exemplary embodiments. Various modifications and changes may be made to an exemplary embodiment without departing from the scope of the present invention and all such modifications are intended to be included within the scope of the present invention.

Synucleinopathies, including Parkinson disease (PD), multiple system atrophy (MSA), pure autonomic failure (PAF) and dementia with Lewy bodies (DLB) are a group of progressive neurodegenerative disorders that affect over 2 million people in the U.S. and lead to progressive disability and death. In some embodiments, these neurodegenerative disorders can be characterized pathologically by analysis of the deposition of phosphorylated α-synuclein within the nervous system. For physicians, diagnostic dilemmas may arise early in the disease course, in patients with atypical symptoms, or in other diseases that may mimic symptoms of synucleinopathies such as progressive supranuclear palsy (PSP), cerebellar ataxia or essential tremor. Some embodiments provide a diagnostic test to (1) help resolve these dilemmas by accurately identifying patients with a synucleinopathy, (2) differentiate between synucleinopathies and (3) be easily accessible and cost effective. In some embodiments, the diagnostic test can facilitate early diagnosis even before motor symptoms emerge, measure target engagement. Some embodiments provide a surrogate endpoint for potential neuroprotective and disease modifying therapies.

Various embodiments, as described herein, provide a diagnostic biomarker for the synucleinopathies, which identifies alpha-synuclein in tissues and organs outside the central nervous system.

In various embodiments provide diagnostic testing of phosphorylated α-synuclein deposition in cutaneous nerves, which is uniquely situated to meet the clinical need for diagnosing a synucleinopathy. A diagnostic test can include: (1) the increasing utilization of skin biopsy by neurologists and primary doctors to make a diagnosis of small fiber neuropathy; (2) the ability to reliably quantify the innervation of the autonomic substructures within the skin; (3) the improvement in confocal imaging, immunofluorescent staining and image analysis to routinely double label and localize protein deposits; and (4) the ability to reliably measure phosphorylated α-synuclein within the nerves supplying these substructures. In some embodiments, the results of the diagnostic test are considered an identification of one or more ‘biomarker’ of synucleinopathy. However, the results of the diagnostic test confirm the presence of phosphorylated α-synuclein within nerve fibers, thereby confirming a diagnosis of synucleinopathy.

Some embodiments provide a pathological test as a diagnostic biomarker for synucleinopathies. In some embodiments, a pathological test method can comprise measuring phosphorylated α-synuclein deposition in standard punch skin biopsies, which can serve as an accurate, precise, sensitive and specific, diagnostic biomarker of synucleinopathy. An effective biomarker identifiable in the skin biopsies (1) provides an accurate diagnosis of alpha-synucleinopathies in clinical practice; (2) differentiates between synucleinopathies, (3) enables assessment of target engagement in the development of disease modifying and neuroprotective therapies; and (4) can accelerate the development of neuroprotective and disease modifying therapies.

Various embodiments provide systems and methods for identifying the deposition of alpha-synuclein in a skin biopsy from a patient and can include determining if the patient has multiple system atrophy (MSA). The methods and systems can include assessing a patient's deposition profile, as compared to a pathological synuclein signature of phosphorylated alpha-synuclein distribution for MSA; and calculating the probability the patient has MSA.

Various embodiments provide systems and methods for combining a patient's clinical features with a patient's deposition profile; and calculating the probability the patient has MSA. The methods and systems can include accessing a data set consisting of a patient's clinical features, a patient's deposition profile, and a pathological synuclein signature of phosphorylated alpha-synuclein distribution; transforming a portion of the data set using a multi-faceted algorithm tool into a result that distinguishes a patient's diagnosis of MSA over PD.

As described herein, within cutaneous autonomic nerve fibers of a skin biopsy tissue, the distribution of the deposition of alpha-synuclein has unique pathological features for MSA, as compared to the other CNS diseases synucleinopathies, such as, for example, but not limited to PD. As described in various embodiments herein, the cutaneous alpha-synuclein distribution pattern, coupled with the assessment of intraepidermal sensory nerve fiber density and dermal autonomic nerve fiber density provide a unique pathological signature in MSA, which can be combined with select clinical features to provide a diagnostic biomarker that is specific for MSA.

Various embodiments provide systems and methods for combining a patient's pathological features into a synuclein signature profile; and calculating the probability the patient has MSA. The synuclein signature profile can include determining the amount of phosphorylated alpha-synuclein in at least one skin biopsy, calculating the distribution of phosphorylated alpha-synuclein synuclein in at least one skin biopsy, and transforming the synuclein signature profile using a multi-faceted algorithm into a result that distinguishes a patient's diagnosis of MSA over PD. In some embodiments, the synuclein signature profile and patient clinic data can be entered into the multi-faceted algorithm, which transforms the profile and data result that distinguishes a patient's diagnosis of MSA or PD, with a probability of greater than 95%.

A simple, sensitive and specific tool for the diagnosis of synucleinopathy is a significant advance in the field. Specifically, the diagnostic tool improves diagnostic accuracy in challenging and complex cases. Diagnostic accuracy is critical in defining appropriate populations for studies targeting disease-modifying strategies: for example, the inclusion of patients with PSP in a PD trial could reduce the power of the study, unbalance the study population, and predispose a treatment to failure. The sensitive diagnostic tool allows early diagnosis, even pre-motor in the case of PD or MSA. Earlier diagnosis facilitates future interventions with neuroprotective and disease modifying therapies at a time when patients are most able to benefit from these interventions. The diagnostic tool can include a biomarker (or a panel of biomarkers) that correlates with disease progression may be used to determine the response to disease modifying and neuroprotective strategies by quantitatively analyzing any change in α-synuclein deposition. The utility of a simple, widely applicable diagnostic tool to diagnose synucleinopathy would aid in the clinical care of patients by allowing for improved prognostic certainty and permit patients to make better life plans

Accordingly, some embodiments provide methods for detection of diagnostic biomarkers of a neurodegenerative disorder in a patient. Such methods can include the steps of obtaining a biopsied skin sample from the patient, performing a dual-immunohistochemical microscopic assay to determine the presence or absence of one or more diagnostic biomarkers in the biopsied sample, and diagnosing a neurodegenerative disorder if one or more diagnostic biomarkers are present. The neurodegenerative disorder can be, but is not limited to, amyloidosis, Parkinson's disease, dementia with Lewy bodies, multiple system atrophy, pure autonomic failure, Alzheimer's disease, Amyotrophic lateral sclerosis, and other synucleinopathies and tauopathies

Some embodiments provide methods of generating a patient-specific diagnostic biomarker profile by quantitative analysis of a biological skin sample from a patient. The methods can include the steps of performing a quantitative neuropathological assessment of sensory and/or autonomic nerve fiber density of at least one of an intra-epidermal nerve, a sweat gland nerve, or a pilomotor nerve, a sub-epidermal nerve, and a dermal nerve.

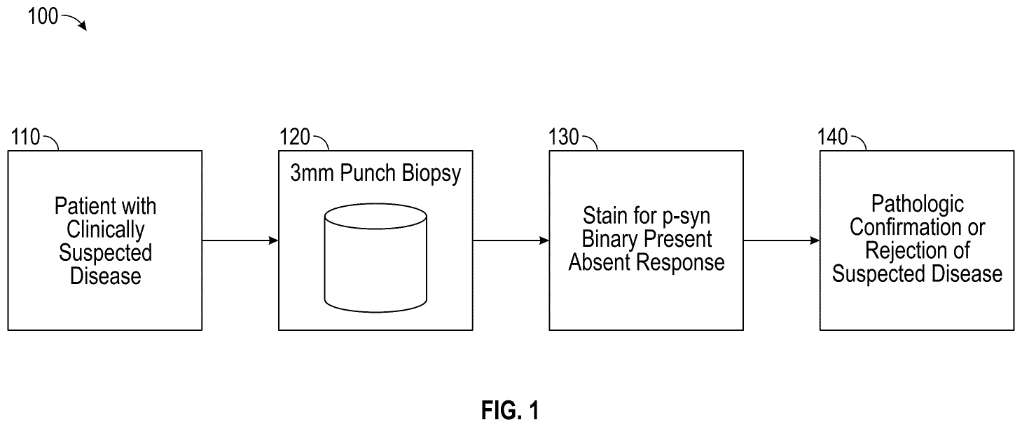

In some embodiments, target proteins in a skin biopsy sample can be tagged by double immunohistochemical staining for analysis of abnormal protein deposition and simultaneous analysis of nerve fiber densities. An exemplary of a method 100 of diagnosing a neurodegenerative disorder is illustrated in FIG. 1. A patient 110 has been diagnosed with one of the synucleinopathies (PD, MSA, PAF and DLB), which has been clinically confirmed by medical experts. One or more punch biopsies 120 are extracted from the patient 110. The punch biopsies are stained using a dual-immunohistochemical staining process 130, which tags one or more target proteins with two fluorescent dyes. The stained punch biopsies are read using a confocal microscope and pathologic confirmation or rejection 140 of the clinically diagnosed neurodegenerative disorder is determined.

Typically, a number of skin biopsies from different regions can be extracted from a patient. The skin biopsies can be fixed in Zamboni solution and stored in cryoprotectant. Frozen tissue sections are cut and six sections per biopsy can be dual immunostained for protein gene product 9.5 (PGP9.5) and phosphorylated α-synuclein. Sections can be washed and immunostained for secondary antibodies using Cy2 and Cy5 (Jackson ImmunoResearch) and viewed by laser scanning confocal microscope (for example: Zeiss Axiocam Apotome2). Biopsies can be reviewed for positive areas of phosphorylated α-synuclein deposition that co-localized with PGP9.5. Regions of interest of the tissue sections can be imaged using confocal Z-stack images in the tissue sections with 3-dimensional co-localization.

More specifically, tissue sections can be washed together in one well of a 12-well plate with tris buffer at room temperature. The tissue sections can then be blocked together in one well of a 96-well round bottom plate with blocking solution at room temperature. After blocking, the tissue sections can be incubated in mouse anti-PSYN and rabbit anti-PGP in a TBS solution at room temperature. After incubation is complete, tissue sections can be washed together in one well of a 12-well plate with tris buffer room temperature. To attach the first probe, the tissue sections can be incubated in anti-mouse biotin and anti-rabbit Cy2 in a TBS solution at room temperature. After incubation is complete, tissue sections can be washed together in one well of a 12-well plate with tris buffer at room temperature. To attach the second probe, the tissue section can be incubated in Cy3 in a TBS solution at room temperature. After incubation is complete, tissue sections can be washed together in one well of a 12-well plate with tris buffer at room temperature. As a result of these preparation steps, the tissue section will be stained, and the target proteins will be tagged with 2 fluorescent dyes (Cy2 and Cy3). The stained tissue samples can be mounted one a slide and then cover-slipped. Tissue sections can be between 0.1 μm thick and 100 μm thick.

Some embodiments provide methods of microscopy analysis of one or more immunostained biomarkers for diagnosis of a neurodegenerative disorder. The stepwise progression through this analytic technique establishes the basis of this method as a diagnostic biomarker of disease diagnosis, and a biomarker of disease severity. In some aspects of the methods, the microscopy analysis can include deconvolution algorithms to identify and quantitate the biomarkers

Imaging analysis identifies nay abnormal protein depositions in the sample. Sample tissue sections are viewed under a florescent microscopy to determine the presence or absence of abnormal protein deposition. In the setting of staining for amyloid deposition, the sample is viewed under polarized light (not immunofluorescent imaging).

The methods can include a determination of intra-neural or extra-neural protein deposition. With dual immunofluorescent imaging, or dual polarized light- and florescent imaging, the deposition of the abnormal protein is determined to be intra-neural (within nerve fibers), extra-neural (outside nerve fibers), or both. Finding is confirmed by digital image acquisition. Digital image acquisition is obtained through use of a confocal microscope, a laser scanning immunofluorescent microscope, or an immunofluorescent microscope with high resolution camera. The imaging of amyloid deposits through a polarized lens is obtained through a light microscope.

Locations of protein deposition are recorded: if intra-neural, specific nerve subtypes are documented (intra-epidermal, sub-epidermal, sudomotor, pilomotor or dermal nerve fibers). If extra-neural, locations are documented (hair follicles, sweat glands, pilomotor muscles, blood vessels, epidermis, dermis).

Analysis for intra-epidermal nerve fiber density is a standardized method to calculate the number of sensory nerves in the epidermal layer (the most superficial layer of the skin). This technique is specific to ‘small nerve fibers’ and does not address any larger nerve fibers that are coated with myelin and does not address autonomic nerve fibers. This technique is can be included in a method because some of the synucleinopathies may selectively damage the small nerve fibers and may provide the ability to distinguish between the disorders (for example, Parkinson disease has a length dependent small fiber sensory neuropathy).

The methods can include measurement of the density of nerve fibers that surround sweat glands. The nerve fibers that surround sweat glands are autonomic nerve fibers (specifically sympathetic cholinergic nerve fibers) and this is one of the common nerves that phosphorylated alpha-synuclein is deposited. Calculating the sweat gland nerve fiber density in some cases can distinguish between different disorders. This technique is often paired with intra-epidermal nerve fiber density. In addition, this technique can quantify the density of nerve fibers that surround sweat glands and quantify the deposition of total alpha-synuclein within these nerve fibers.

Examples and techniques useful in this analysis can be found in the following article Gibbons C H, Illigens B M, Wang N, Freeman R.: Quantification of sweat gland innervation: a clinical-pathologic correlation. Neurology 2009; 72:1479-1486.

The methods can include techniques that quantify the number of nerve fibers within pilomotor muscles (the muscles in the skin that cause goosebumps). These nerve fibers are autonomic nerve fibers (specifically they are sympathetic adrenergic nerve fibers). This is another area where phosphorylated alpha-synuclein is detected. This technique can quantify the density of nerve fibers with pilomotor muscles and quantify the deposition of total alpha-synuclein within these nerve fibers.

The methods can include techniques that quantify the number of nerve fibers that surround blood vessels. These nerve fibers are autonomic nerve fibers (specifically they are sympathetic vasomotor nerve fibers). This is another area where phosphorylated alpha-synuclein is detected. This technique can quantify the density of nerve fibers around blood vessels and quantify the deposition of total alpha-synuclein within these nerve fibers.

Examples and techniques useful in analysis vasomotor nerve fiber density and the density of nerve fibers with pilomotor muscles can be found in the following article: Gibbons C H, Wang N, Freeman R. Capsaicin induces degeneration of cutaneous autonomic nerve fibers. Annals of neurology 2010; 68:888-898.

The methods can include techniques that quantify the density of nerve fibers in the region just below the epidermal layer. These nerve fibers are sensory nerve fibers. This is another area where phosphorylated alpha-synuclein is occasionally detected.

The methods can include an amalgamation of the techniques that are listed above and merge the density of these fibers in the skin into a total calculation.

The methods can include quantitative analysis of protein deposits. For intra-neural proteins, the deposits are counted as discrete samples normalized to the density of the nerve fiber subtype. For extra-neural proteins, the deposits are counted as discrete samples normalized to the area/volume of tissue analyzed.

Quantitative analysis of nerve fiber densities within the skin biopsy, including one, or more, of the following: intra-epidermal nerve fiber density, sweat gland nerve fiber density, pilomotor nerve fiber density, vasomotor nerve fiber density, sub-epidermal nerve fiber density, and dermal nerve fiber density.

The methods can include the staging of disease severity through comparison of both protein deposition and nerve fiber degeneration. The quantitative staging of protein deposition is defined against normative values based on age and gender. Diagnostic staging occurs in the presence of an abnormal protein (for example, phosphorylated alpha-synuclein) that is not present in non-disease samples. The presence of the abnormal protein determines the diagnosis of disease.

The quantitative staging of severity occurs by quantifying the volume of protein against normative and disease staged values to define the severity of the disease in a particular tissue sample. The quantitative staging of nerve fiber density (intra-epidermal, sub-epidermal, sudomotor, pilomotor or dermal nerve fibers) against age and gender-controlled values to determine the neural damage associated with the disease state.

These methods provide several superior advantages and benefits. First, the identification of biomarkers provides more comprehensive and less expensive diagnosis of injury severity than existing diagnostic devices such as computed tomography (CT), magnetic resonance imaging (MRI) single photon positron emission computed tomography (SPECT) and dopamine transporter (DaTscan) imaging. In additions, the methods, described herein, provide quantitative detection and nerve fiber subtype assessment of damage to the peripheral nervous system (i.e. quantified axonal damage of specific nerve fiber subtypes).

As described above, the methods provide the step of correlating the presence or amount of one or more neural protein(s) with the severity and/or type of nerve cell injury. The amount of a neural proteins, peptides, fragments, derivatives or the modified forms, thereof, directly relates to severity of nerve tissue injury as more severe injury damages a greater number of nerve cells which in turn causes a larger amount of neural protein(s) to accumulate in the biological sample (e.g., skin).

In these methods, an abnormal protein can be one of phosphorylated alpha-synuclein, total alpha-synuclein, tau protein, TDP-43, microtubule associated protein, and amyloid protein. The methods provide a step of detecting and quantify nerve tissue through use of at least one additional neural antibody marker selected from the group consisting of: protein gene product 9.5 (PGP 9.5), vasoactive intestinal peptide (VIP), tyrosine hydroxylase (TH).

Examples and techniques useful in this analysis can be found in the following article: Gibbons C H, Wang N, Kim J Y, Campagnolo M, Freeman R. Skin Biopsy in Evaluation of Autonomic Disorders. Continuum (Minneap Minn). 2020 February; 26(1):200-212. PMID: 31996629.

Other examples and techniques useful in this analysis can be found in the following article: Gibbons C H, Garcia J, Wang N, Shih L C, Freeman R. The diagnostic discrimination of cutaneous α-synuclein deposition in Parkinson disease. Neurology. 2016 Aug. 2; 87(5):505-12. PMID: 27385742.

More examples and techniques useful in this analysis can be found in the following article: Wang N, Gibbons C H, Lafo J, Freeman R. α-Synuclein in cutaneous autonomic nerves. Neurology. 2013 Oct. 29; 81(18):1604-10. PMID: 24089386.

To define the test accuracy and precision of skin biopsy detection of phosphorylated α-synuclein, a blinded quantitation of phosphorylated α-synuclein deposition from skin biopsies can accurately detect a diagnosis of synucleinopathy in individuals with PD, MSA, PAF and DLB clinically confirmed by disease experts. The results, from performing repeated quantitative measurements of skin biopsies from individuals across the synucleinopathy spectrum with a diagnosis of PD, MSA, PAF or DLB (as confirmed by the disease experts), over several time points, can be mapped to the clinical diagnosis and compared for accuracy and precision of detection.

To define the sensitivity and specificity of skin biopsy detection of phosphorylated α-synuclein deposition for the diagnosis of synucleinopathies, results from performing repeated quantitative measurements of skin biopsies from control subjects over several time points, can be mapped with and compared to the results from the cohort of individuals with a diagnosis of synucleinopathy.

The results from the cohort can be further analyzed for distributions based on sex and/or age. Other demographic information can be collected for the cohort and the results from the cohort can be further analyzed for distributions based on one or more aspects of the demographic information. Risk factors for a specific diagnosis of synucleinopathy can be identified by the cohort comparisons of results and distributions, which can be integrated into data analysis for identifying a specific disorder of synucleinopathy.

In some embodiments, quantitation of phosphorylated α-synuclein within skin biopsies (by biopsy location and within specific nerve fiber subtypes) can differentiate between the synucleinopathies (PD, DLB, MSA and PAF). Some embodiments provide a validated skin biopsy detection of phosphorylated α-synuclein by defining the (1) accuracy & precision, (2) sensitivity and specificity of the test across all synuclein disease states (PD, MSA, PAF and DLB) and (3) differentiate between the different synucleinopathies.

A system for reading the degree of immunofluorescence and reporting can rates the abnormalities of identified proteins from 0 to 4+. This system gives referring doctors a measure of the severity of the disease. In some configurations, the systems can identify which structures within the skin are most affected by synuclein, which allows differentiate between the five different forms of synucleinopathy.

The inclusion of a large data set into the phosphorylated alpha-synuclein database can provide key risk determinants of disease. Through inclusion of these factors with the tissue results, the system can specifically identify the underlying disease, the underlying disease course, and the potential future outcomes. Specific key factors include age, gender, race, medical history, family history, medication use, symptoms of neurodegenerative disease and age of symptom onset. An example of a patient questionnaire to collect many of these key factors is included herein.

This clinical questionnaire allows the collection large amounts of clinical data, which is entered an algorithm that incorporates the clinical symptoms and the pathological findings to make a more accurate diagnosis. Some embodiments provide a multi-faceted algorithm diagnostic tool. Data collected by the inventors indicate that many neurodegenerative diseases have a common thread which is the development of proteinaceous inclusions within the nerves. The multi-faceted algorithm diagnostic tool inputs the results, from the identification methods describe herein, into a standard, reproducible, sensitive and specific set of algorithms for identifying these aberrant proteins. Simply visualizing with the human eye then quantifying these proteins is not sufficient to aid in the clinical diagnosis. Since a significant amount of heterogeneity exists in clinical patient populations, each disease has multiple mechanisms at play, which need to be considered in the clinical diagnosis.

The multi-faceted algorithm diagnostic tool may use the presence or absence of these proteins, however, it emphasizes an algorithmic model to overlay the results from a series of clinical questions with the exact and precise anatomic localization to achieve a very high sensitivity and specificity for the clinician. In addition, the a multi-faceted algorithm diagnostic tool accesses data (see Table 1) then determines risk factors that are used in the calculates to yield the clinical diagnosis.

TABLE 1
Examples of Data Sets Entered into the
Multi-faceted Algorithm Diagnostic Tool

The Clinical Questionnaire
Presence or Absence of aberrant proteins
involved in neurodegenerative diseases
Localization of protein deposition,
which is entered into an algorithm that
segregates deposition to the different
nerve structures within the skin
Analysis of dermal and epidermal nerves.

Using the algorithms discussed herein, each of these combinations of variables will predict with a high degree of sensitivity and specificity which disease is present. In some embodiments, the system for quantifying the aberrant proteins (Table 2) correlates with the stage and severity of disease. This technique has already proven to be of significant value in clinical research trials. Pharma companies can use this data to evaluate objectively whether their drug has an impact on the protein deposition will continue to be important as more neuroprotective agents are discovered.

TABLE 2

| | Examples of Proteins | Disorder/Disease |
|---|---|---|
| Protein | that may be included | Mutated Protein |
| Groups | in the Group | Biomarker |
| Syn | Alpha-synuclein, SNCA, | Parkinson's, |
| | NACP, PARK1, PARK4, | Dementia with Lewy |
| | PD1, | Bodies, |
| | | Multiple System Atrophy, |
| Tdp | TARDBP, ALS10, TDP-43, | Armyotrophic Lateral |
| | TAR DNA binding protein | Sclerosis, |
| | | Alzheimer's |
| Tau | MAPT, DDPAC, FTDP-17, | Progressive Supranuclear |
| | MAPTL, MSTD, MTBT1, | Palsy, |
| | MTBT2, PPND, PPP1R103, | Alzheimer's, |
| | TAU | Parkinson's, |
| | | Tauopathy |
| Amyloid | Group of 37 proteins, | Alzheimer's (Aβ) |
| | which includes some | Parkinson's (AαSyn) |
| | of the examples | PSP (ATau) |
| | in this table | Danish Dementia (ADan) |
| | | British Dementia (ABri) |
| Frataxin | FXN, CyaY, FA, FARR, | Friedreich's Ataxia |
| | FRDA, X25, frataxin | |
| Huntington | Huntington exon 1 | Huntington's Disease |

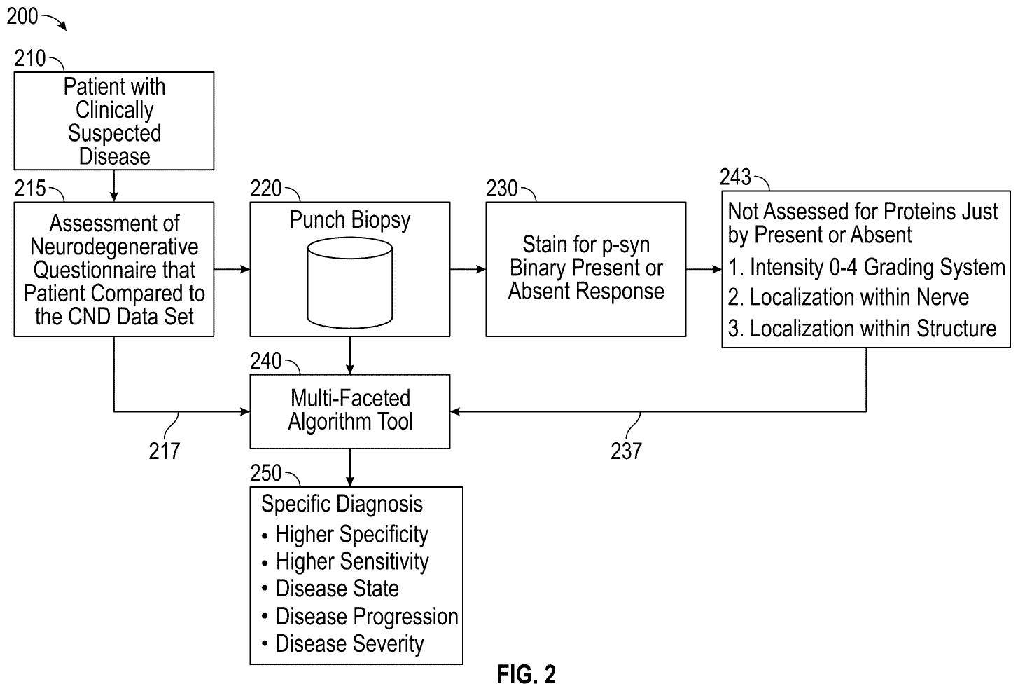

For example, in FIG. 2 a system 200 of determining a diagnosis of a neurodegenerative disorder using detection of phosphorylated α-synuclein from skin biopsies and other risk factors is illustrated. A patient 210 has been diagnosed with one of PD, MSA, PAF and DLB, which has been clinically confirmed by medical experts. The patient 210 and medical expert fill out a patient questionnaire 215, which includes key factors, include age, gender, race, medical history, family history, medication use, symptoms of neurodegenerative disease and age of symptom onset. In addition, the patient questionnaire 215 can include a medical expert portion, which collects the data and analysis from the clinical diagnosis. The key factors 217 (which can include medical diagnosis and other information from the medical expert) from the questionnaire 215 is entered into the multi-faceted algorithm tool 240.

The punch skin biopsy sample 220 are taken from the patient and sent to a lab. At the lab, the skin biopsy sample 220 are processed and stained using a dual-immunohistochemical staining process 230, as described herein. The stained punch skin biopsy sample 220 is analyzed 240 using a confocal microscope and pathologic confirmation or rejection of the clinically diagnosed neurodegenerative disorder is determined, as well as intensity of the identified abnormal proteins, the amount of abnormal proteins within nerves and within other structures in the skin. The data 247 from this analysis is entered into the multi-faceted algorithm tool 240.

The multi-faceted algorithm tool 240 accesses the patient's key factors 217 and data 247 from the analysis of the skin biopsy 220. The tool 240 uses the key factors 217 and data 247 to determine a preliminary diagnosis. The tool 240 compares the preliminary diagnosis (including the key factors 217 and data 247) to the key factors and data of a mixed population of patients diagnosed with various neurodegenerative disorders and patients without a disorder (control) to confirm or deny the preliminary diagnosis. If the preliminary diagnosis is confirmed, the tool 240 compares the key factors 217 and data 247 to the key factors (including demographics) and data of biopsy analysis from a population of patients diagnosed with the preliminary diagnosis to determine the state of, the progression of, and severity of the patient's diagnosis. The tool 240 outputs a patient diagnosis 250 of a specific neurodegenerative disorder, including the state of, the progression of, and severity of the specific disorder.

If the tool 240 denies the preliminary diagnosis, the tool 240 analysis the key factors 217 and data 247, as compared to the key factors and data of a mixed population of patients to determine if a diagnosis of a different neurodegenerative disorder is plausible. If it is plausible, the tool 240 outputs a patient diagnosis 250 of an unconfirmed neurodegenerative disorder and a plausible alternative neurodegenerative disorder. If it is not plausible, the tool 240 outputs a patient diagnosis 250 of an unconfirmed neurodegenerative disorder and no information regarding an alternative neurodegenerative disorder.

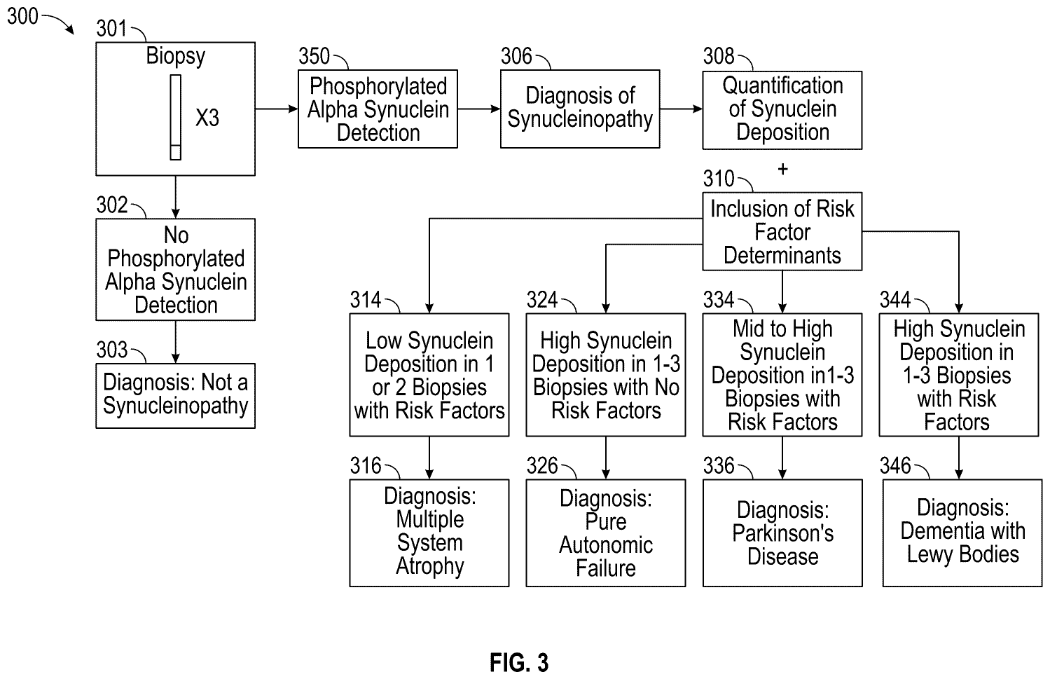

Moving to FIG. 3, a flow chart illustrates an exemplary method 300 that can be executed in a multi-faceted algorithm tool. One of more skin biopsy samples 301 are taken from a patient. The samples are stained using a dual-immunohistochemical staining process, as described herein. The stained samples are then analyzed. If no phosphorylated synuclein 302 is detected, then the diagnosis is no synucleinothy disorder 303 identified for the patient. If phosphorylated synuclein 302 is detected, then the samples 301 are analyzed to determine a diagnosis 306 of a synucleinothy disorder. Quantification data 308 of the synuclein disposition is determined using techniques described herein, or another appropriate technique known to one of skill in the art. The quantification data 308 is input into the multi-faceted algorithm tool, which combined with risk factors 310 demographic information of the patient and may include test results and a diagnosis from a medical expert. The risk factors 310 can be obtained through a patient questionnaire, as described herein.

The multi-faceted algorithm tool analyzes the quantification data 308 and the risk factors 310 to determine a diagnosis. If the tool determines a result 314 that data 308 has low synuclein deposition in one or two samples 301 and some disorder factors matched the risk factors 310 of the patient, then the diagnosis 316 is multiple system atrophy.

If the tool determines a result 324 that data 308 has high synuclein deposition in one to three samples 301 and no disorder factors matched the risk factors 310 of the patient, then the diagnosis 326 is pure autonomic failure.

If the tool determines a result 334 that data 308 has mid to high synuclein deposition in one to three samples 301 and some disorder factors matched the risk factors 310 of the patient, then the diagnosis 336 is Parkinson's disease.

If the tool determines a result 344 that data 308 has high synuclein deposition in one to three samples 301 and some disorder factors matched the risk factors 310 of the patient, then the diagnosis 346 is dementia with Lewy bodies.

The description supporting FIG. 3 is merely an example of the predictive ability of a system comprising the multi-faceted algorithm tool. Other predictive equations and algorithms can be used to diagnosis other neurodegenerative disorders, such as, for example, Alzheimer's disease and Progressive Supranuclear Palsy. In addition, the multi-faceted algorithm tool can determine, including the state of, the progression of, and severity of the specific neurodegenerative disorder, as discussed in FIG. 2. The methods of the multi-faceted algorithm tool, as described in FIG. 3, can be employed but the multi-faceted algorithm tool 240 of FIG. 2.

The use of the techniques and the multi-faceted algorithm tool described herein can be used as a predictive tool. The data to date clearly shows that most of these diseases begin years or decades before diagnosis. An early detection program can identify patients who possess the proteins but are asymptomatic. This data suggests that as neuroprotective drugs become available, the use of these predictive techniques, to identify patients earlier in the course of their disease or even before they are symptomatic, prevent the development of the disease.

The results, as illustrated herein, indicate that phosphorylated alpha-synuclein increases over time. The ability to quantify the synuclein deposition over time provides predictive modelling of disease outcomes, potential time frames of disease development, and therefore allows predictive modeling of pre-symptomatic detection. The early detection program can incorporate phosphorylated alpha-synuclein deposition and disease modeling into the predictive population testing.

Several key factors of phosphorylated alpha-synuclein deposition have been determined that distinguish between the different diseases (Parkinson's disease, multiple system atrophy and dementia with Lewy bodies). Greater deposition of phosphorylated alpha-synuclein within multiple biopsies is most common in dementia with Lewy bodies. Very low levels of phosphorylated alpha-synuclein in few biopsies suggest multiple system atrophy. Moderate to high levels of phosphorylated alpha-synuclein in some biopsies suggests Parkinson's disease. These results can be entered into the multi-faceted algorithm diagnostic tool, as discussed below.

In some examples, REM disorder diagnosis can be an indicator of increase risk of developing ALS or PD later in a patient's life. A detected phosphorylated alpha-synuclein in skin biopsies of REM sleep behavioral disorders and most of these will go on to develop Parkinson's disease over 15 years. Patients that have phosphorylated alpha-synuclein and REM sleep behavioral disorder will have more rapid disease progression and more rapid conversion to a clinically apparent synucleinopathy. An early detection program for these patients is important for early intervention and treatment.

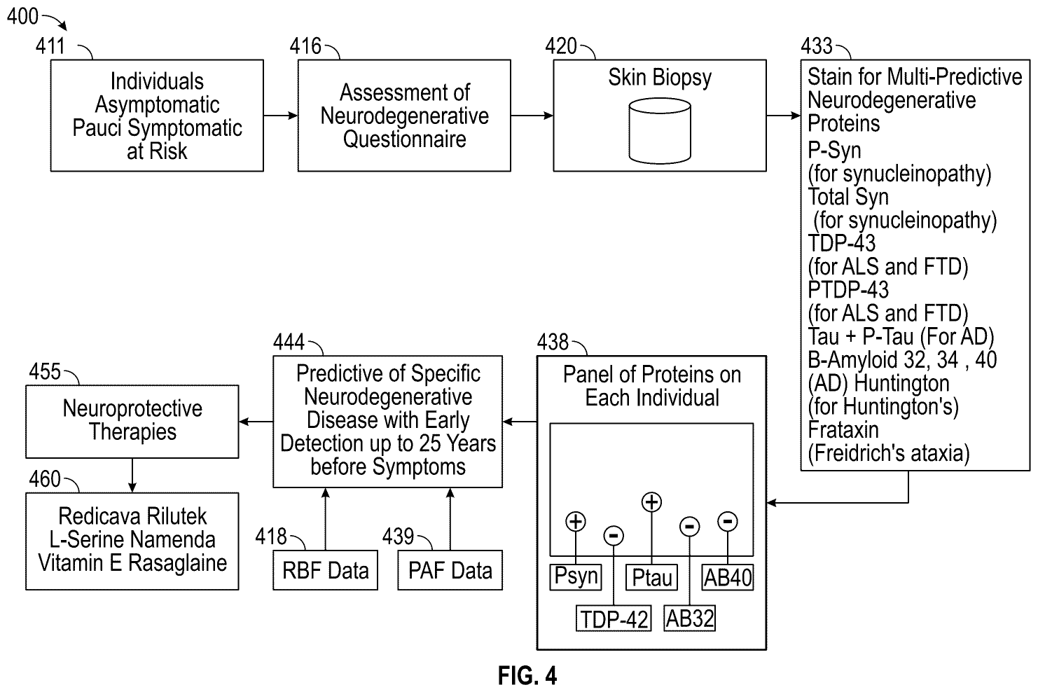

With reference to FIG. 4, a flow chart illustrates an exemplary method 400 that can be executed in a multi-faceted algorithm tool to define disease and optimize treatment. Asymptomatic patient 411 at risk for disease can be identified. A patient 411 may be diagnosed with one of PD, MSA, PAF and DLB, which has been clinically confirmed by medical experts. The patient 411 and medical expert fill out a patient questionnaire 416, which includes key factors, include age, gender, race, medical history, family history, medication use, symptoms of neurodegenerative disease and age of symptom onset. In addition, the patient questionnaire 416 can include a medical expert portion, which collects the data and analysis from the clinical diagnosis. The key factors 418 (which can include medical diagnosis and other information from the medical expert) from the questionnaire 416 is entered into the multi-faceted algorithm tool 444.

The punch skin biopsy sample 420 are taken from the patient and sent to a lab. At the lab, the skin biopsy sample are processed and stained using a multi-plex immunohistochemical staining process 433 for multiple proteins (including, but not limited to, P-SYN, total SYN, TDP-43, PTDP-43, Tau, P-Tau, β-Amyloid 32, β-Amyloid 34, β-Amyloid 40, Huntington, Frataxin) to produce a panel of biomarkers for a multitude of neurodegenerative disorders. The panel 438 of multi-plex immunohistochemical stained punch skin biopsy sample 420 is analyzed to quantitate the amount of abnormal proteins within nerves and within other structures in the skin. After the results from the panel of biomarkers 438 are quantitatively determined, the resulting panel data 439 is entered into the multi-faceted algorithm tool 444. For example, the panel of biomarkers 438 illustrates a positive for Psyn, and Ptau but a negative for TDP-42, AB32, and AB40, which in this example is measured quantitatively and input as panel data 439 into the multi-faceted algorithm tool 444.

In some embodiments, the multi-faceted algorithm tool 444 can employ one or more of the methods described in FIG. 3.

Based in the key factors 418 and the panel data 439 for the patient 411, the multi-faceted algorithm tool 444 determine a predictive diagnosis of a neurodegenerative disorder for the patient 411. The predictive diagnosis can be determined by running analysis using a database of a population of patients and controls, as described for FIG. 2.

The predictive diagnosis can include one or more neuroprotective therapies 455, which are designed to delay the onset of, limit the degree of the onset of, or prevent the onset of the predictive neurodegenerative disorder for the patient 411. The neuroprotective therapies 455 can include treating 460 the patient 411 with a therapeutic active amount of a drug, natural product, vitamin, and combinations thereof. The treating 460 the patient 411 with one or more doses of the therapeutic amount of an active ingredient, effectively delay the onset of, limit the degree of the onset of, or prevent the onset of the predictive neurodegenerative disorder for the patient 411. The treating 460 the patient 411 can include, but are not limited to, treatment strategies that include the administration of one or more of Redicava, Rilutek, L-serine, Namenda, Vitamin E and Rasagaline.

In some embodiments, if a protein biomarker from the panel 438 is detected, then there is a predictive diagnosis of disease up to 25 year prior to symptoms. For example, in REM sleep behavioral disorder (RBD) and in PAF can be predictive of a future onset of a neurodegenerative disorder in the patient 411 in the future.

In some embodiments, an early detection program can include taking a patient's simple skin biopsy test, for example, every 5 years beginning at age 50 (like a preventative colonoscopy), then using multi-faceted algorithm diagnostic tool to determine if any of these proteins are available to prevent Parkinson's, Alzheimer's and ALS.

Examples: the following examples are non-limiting and are included to further describe the invention and to provide some exemplary data.

### Example 1

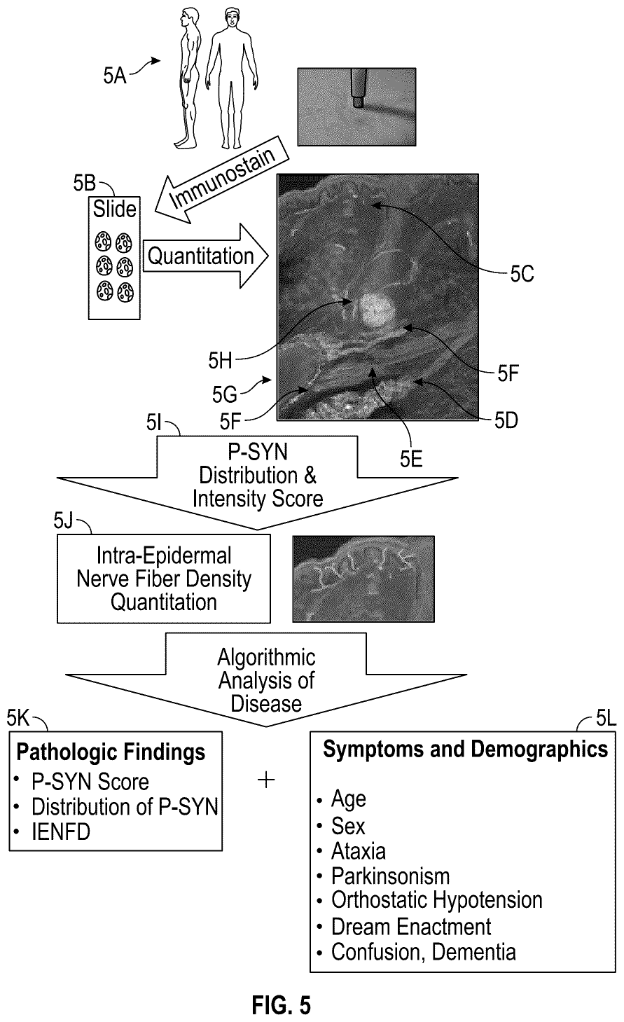

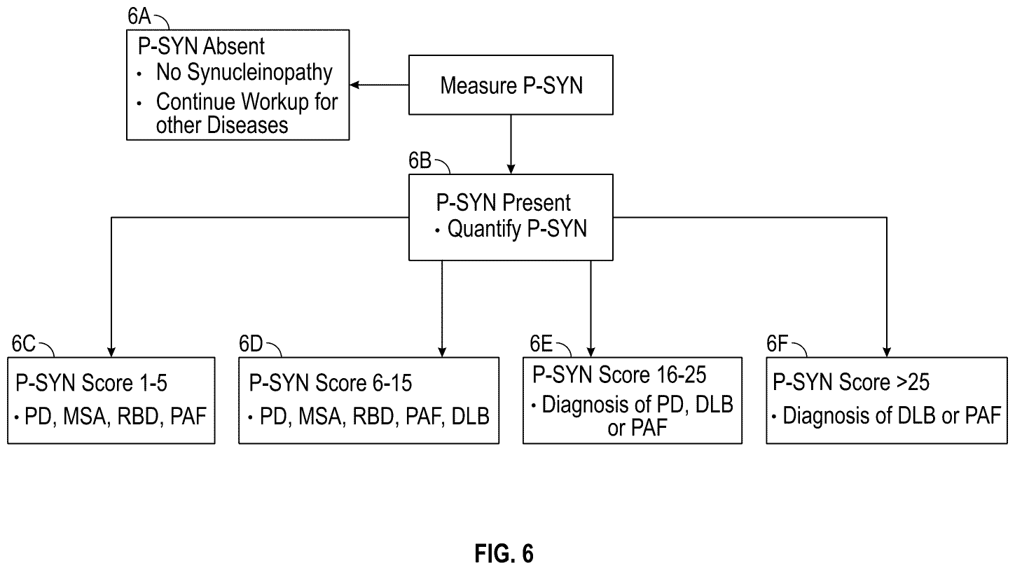

A non-limiting example of a multi-faceted algorithm tool is described in in the following paragraphs and is illustrated in FIGS. 5 and 6.

From 3 biopsy samples (5A) from a patient, the amount, the intensity and the distribution of co-localized phosphorylated alpha-synuclein deposits within nerve fibers within the skin biopsy sections are determined. Each biopsy will have 6 tissue sections immunostained for both protein gene product 9.5 and phosphorylated alpha-synuclein (5B).

In addition, the intra-epidermal nerve fiber density (IENFD) in the biopsy samples are reported by quantifying the number of fibers stained with protein gene product 9.5 that clearly cross the dermal-epidermal junction. The dual immunostaining process, as described herein, is necessary to confirm that any positive phosphorylated alpha-synuclein samples are contained within a nerve fiber stained by protein gene product 9.5, and not simply an artifact that occurs due to background immunofluorescence uptake.

The quantitation of phosphorylated alpha-synuclein is calculated within each tissue section and the type of nerve fiber (sub-epidermal plexus nerve fibers (5C), sweat gland nerve fibers (5D), pilomotor nerve fibers (5E), nerve bundles, which are tracks of nerve fibers travelling together within the deeper dermal tissue (5F), hair follicle nerve fibers (5G), and vasomotor nerve fibers 5H)) containing phosphorylated alpha-synuclein is recorded.

The quantitation of phosphorylated alpha-synuclein is further reported by distribution within the tissue sections (5I):

Distribution Score 0: No phosphorylated alpha-synuclein present

Distribution Score 1: A single nerve fiber containing phosphorylated alpha-synuclein is detected.

Distribution Score 2: Two or more nerve fibers contain phosphorylated alpha-synuclein, but not the majority of tissue sections.

Distribution Score 3: Nerve fibers containing Phosphorylated alpha-synuclein are detected within at least 4 of 6 tissue sections.

Distribution Score 4: Nerve fibers containing phosphorylated alpha-synuclein are detected within every tissue section.

The quantitation of phosphorylated alpha-synuclein is further reported by the intensity of distribution within the tissue sections, and is reported by the single maximally intensively stained fiber detected within the biopsy.

Intensity Score 0: (No phosphorylated alpha-synuclein present, automatically noted with all Distribution Scores of 0.

Intensity Score of 1: Phosphorylated alpha-synuclein is detected on skin biopsy, but is faint enough that it cannot be seen when viewing image under a dual immunofluorescent filter (showing both phosphorylated alpha-synuclein and protein gene product 9.5 simultaneously).

Intensity Score of 2: Phosphorylated alpha-synuclein is detected on skin biopsy and can be seen when viewing image with a dual immunofluorescent filter (showing both phosphorylated alpha-synuclein and protein gene product 9.5 simultaneously) but is not immediately apparent under dual filter viewing.

Intensity Score of 3: Phosphorylated alpha-synuclein is immediately visible when viewing image with a dual immunofluorescent filter (showing both phosphorylated alpha-synuclein and protein gene product 9.5 simultaneously).

The combination of distribution and intensity are both quantified for each biopsy from each patient. The multiplied score of distribution X Intensity score then provides a sum score for each biopsy (range 0-12 score). The sum of all 3 biopsy scores are then provided for a total score (with a potential range of 0-36). The locations of the phosphorylated alpha-synuclein are provided as an additional note along with the sum score.

The number of nerve fibers per tissue section is reported, combined with the other tissue sections, and divided by the total length of tissue measured (typically −3 mm of tissue). The intra-epidermal nerve fiber density is reported as the number of fibers per millimeter of tissue (5J).

As described above, the following pathological information has been quantified and reported for each skin biopsy (5K): 1. Phosphorylated alpha-synuclein score, 2. Distribution of phosphorylated alpha-synuclein deposition by fiber types, and 3. Intra-epidermal nerve fiber density.

The following clinical information is acquired from the patient (5L): Age, Sex, Ataxia, Parkinsonism, Orthostatic hypotension, Dream enactment, and Confusion/dementia. The following weighted values are included to determine the relative likelihood of the underlying diagnosis.

As illustrated in FIG. 6, the phosphorylated alpha-synuclein total score is the initial quantitative measure to determine the specific neurodegenerative disorder or lack thereof.

### Step 1:

6A) P-SYN Score 0: Diagnosis of synucleinopathy<5%. Alternative diagnoses (non-synucleinopathy) should be considered.
6B) P-SYN Scores>0—Diagnosis of Synucleinopathy (MSA, PAF, PD, DLB, RBD)

### Step 2:

6C) P-SYN Scores Low (1-5): Diagnosis of PD, MSA, RBD or PAF phenoconverting to MSA>80%. Unlikely to be DLB unless extremely early in disease and asymptomatic.
i) With reduced IENFD at distal leg, or distal leg+distal thigh diagnosis is likely PD (85%). However, if evidence of phosphorylated alpha-synuclein within subepidermal plexus then diagnosis of PD is reduced to <30%, with MSA>70%.
ii) Without reduced IENFD at any site, with age<65, and with phosphorylated alpha-synuclein deposition within subepidermal plexus diagnosis is likely MSA (>90%).
iii) With normal IENFD at all sites, and history of dream enactment without hallucinations or tremors then diagnosis of RBD>90%.
6D) P-SYN Scores Medium (6-15): Possible diagnosis of PD, MSA, RBD, PAF, or DLB.
i) For age>70 without ataxia or parkinsonism, diagnosis of MSA (<10%)
ii) For age<70 without reduced IENFD, and phosphorylated alpha-synuclein present in sub-epidermal plexus MSA diagnosis>90%
iii) With reduced IENFD at distal leg, or distal leg+distal thigh diagnosis is likely PD or DLB (>90%).
iv) With normal IENFD at all sites, without parkinsonism or ataxia, and history of dream enactment without hallucinations or tremors then diagnosis of RBD>90%.
v) If presence of confusion or dementia, diagnosis is DLB>90%
vi) If presence of orthostatic hypotension is also noted without parkinsonism, ataxia, or confusion/dementia then diagnosis is PAF>95%

6E) Scores High (16-25): Likelihood of diagnosis of PD, DLB or PAF>90%
i) With presence of reduced IENFD at distal leg or distal thigh diagnosis is PD or DLB>95%
ii) With normal IENFD, without ataxia, parkinsonism or confusion/dementia, and with orthostatic hypotension, diagnosis is PAF>95%

6F) Scores Very High (>25): Likelihood of diagnosis of DLB or PAF>90%.
i) With history of orthostatic hypotension without parkinsonism or confusion/dementia diagnosis is PAF>95%
ii) With history of confusion/dementia or parkinsonism diagnosis if DLB>95%

Additional variables of phosphorylated alpha-synuclein deposition within sudomotor, pilomotor or vasomotor nerve fibers may further refine the subtype of neuropathy that is likely to be present.

Some of the configurations of the systems and the methods described herein can be optimized and automated to improve efficiency and reduce potential errors through automation. The technical aspects of automation involve a number of key steps in the processing, sectioning, staining, imaging and analysis of data. Each of these parts of automation will be described individually.

Processing: Tissue processing is the critical first step for completion for all of the tests. The tissue is received, coded and accessioned into the database, which includes a tissue tracking program. After accessioning, tissue is place into the assigned conveyor system where it moves into the washing and cryoprotection stages as standard preset time frames. Tissue is then shuttled into refrigerated storage for optimal tissue protection while awaiting section.

Sectioning: By freezing the specimen in dry ice, moving it onto an automated stage, and sectioning the tissue frozen sections (for example, in a range from 0.1 to 100 micrometer thick) using our semi-automated cryostat sectioning system. Manual supervision maybe required at this stage for appropriate tissue orientation and cutting, while tissue is transferred into storage medium before staining.

Staining: A mobile platform can move tissue sections through the steps of incubation, washing and staining using specialized plates with microfiltration membranes at the base to allow movement of fluids into, and out of, the incubation tray. Tissue sections are manipulated less than during manual staining with this process and therefore have improved visualization by microscopy. Completed tissues are then mounted on slides and stored in refrigerated chambers until moved into the imaging stages.

Imaging: Once the tissues have been stained, they are shuttled into an automated confocal microscopy imaging system with laser scanning technology to acquire permanent records of the tissue through high resolution Z-stack imaging. Tissue imaging is completed using automated microscope stages driven through technologist assisted section identification.

Data Analysis: Once tissue imaging has been completed the results are transferred into the algorithmic quantification for florescent co-localization using to define regions of interest. Nerve fibers are stained by protein gene product 9.5 and appear as a single monochromatic output in the light spectrum of 520-560 nanometers. The secondary imaging (such as that used with synuclein testing) is measured within the regions defined by protein gene product 9.5 and will appear in the 635-700 nanometer light spectrum. Regions of overlap between the two sections create a new wavelength of visible light in the 560-630 light spectrum and serve to notify the pathologist of the regions of interest. Further algorithmic learning will determine the likelihood of the result being a true positive based on the location within associated dermal structures of interest (e.g. the AI recognition of the structure of interest as a nerve bundle, blood vessel, sweat gland or pilomotor muscle).

Using a multi-faceted algorithm tool, as described herein, a final automated outcome will incorporate clinical data (age, gender, sex, and other responses to the clinical questionnaire) into the image result to provide an algorithm of risk by disease entity of the individual. The final automated output will be transferred into the patient clinical report. In addition, the final automated output can include a treatment plan designed specifically for the patient.

### Example 2

A series of studies were conducted on individuals with synucleinopathies to assess (1) the number of biopsies required, (2) accuracy, (3) precision, (4) sensitivity, (5) specificity and (6) differentiation between synucleinopathies through detection of phosphorylated α-synuclein.

The rigorous protocols have continued to report a high degree of accuracy, reliability and reproducibility in detection of phosphorylated α-synuclein from skin biopsies. To date, in academic laboratories a total of 336 patients with clinically evident synucleinopathy have been studied. These studies include 241 patients with PD, 31 patients with DLB, 36 patients with MSA and 28 patients with PAF have been reported across 15 different studies. In addition, 238 healthy controls and 74 disease controls have been studied. Although the disease severity, specific biopsy sites, number of biopsies, size of biopsies and number of tissue sections has varied across labs and studies, an analysis of the data in total reveals that the pathological phosphorylated α-synuclein is detected in 88% of PD, 100% of DLB, 77% of MSA and 100% of PAF patients. No phosphorylated α-synuclein has been detection in any of the 238 healthy control samples and none of the 74 non-synucleinopathy disease controls. A summary of the 15 studies (with the synucleinopathy patients reported as a single disease group) is provided in Table 3. The results of this analysis highlight 100% specificity and a very high sensitivity and accuracy.

Furthermore, in REM sleep behavioral disorder (RBD), a disorder without clinically evident motor features, a total of 71 patients have been studied. Phosphorylated α-synuclein is detected within punch skin biopsies of 53/71 (74%) of individuals with RBD without clinically apparent synucleinopathy.

Finally, in 11 autopsy confirmed synucleinopathy cases, and 5 non-synucleinopathy autopsy confirmed cases we successfully confirmed the quantitation of alpha-synuclein deposition from the skin of all 11 patients with autopsy confirmed synucleinopathy.

TABLE 3

| Analysis of 15 studies | Result |
|---|---|
| True Positive | 296 |
| False Positive | 0 |
| False Negative | 40 |
| True Negative | 312 |
| Sensitivity | 88.1% |
| Specificity | 100% |
| Positive predictive value | 100% |
| (not a population-based estimate) | |
| Negative predictive value | 88.64% |
| (not a population-based estimate) | |
| Accuracy | 93.86% |
| (not a population-based estimate) | |

Efforts to distinguish between synucleinopathies have included demographic information, assessments of cognition and autonomic function, with some moderate improvements over clinical diagnosis alone. However, significant overlap between disorders is noted and definitive diagnosis is not ascertained until autopsy. Improved differentiation between synucleinopathies is an unmet clinical need.

In conclusion, phosphorylated α-synuclein is detected within punch skin biopsies across all stages of the disease spectrum, from the premotor manifestations of RBD to autopsy confirmation of synucleinopathy in PD, DLB and MSA. These findings strongly support the need to develop this research technique into a commercially available test for synucleinopathies where physicians and patients can benefit from these scientific advances.

A series of studies were conducted on individuals with synucleinopathies to assess (1) the number of biopsies required, (2) accuracy, (3) precision, (4) sensitivity, (5) specificity and (6) differentiation between synucleinopathies through detection of phosphorylated α-synuclein. To assess the number of biopsies required we performed five (5) skin biopsies from different regions in 45 patients with clinically confirmed synucleinopathy to evaluate the number of biopsies required to optimize testing. Skin biopsies were fixed in Zamboni solution and stored in cryoprotectant. Frozen tissue sections were cut and six sections per biopsy were dual immunostained for protein gene product 9.5 (PGP9.5) and phosphorylated α-synuclein. Sections were washed and immunostained for secondary antibodies using Cy2 and Cy5 (Jackson ImmunoResearch) and viewed by laser scanning microscope (Zeiss Axiocam Apotome2). Biopsies were reviewed for positive areas of phosphorylated α-synuclein deposition that co-localized with PGP9.5. Regions of interest were imaged using confocal Z-stack images in 0.3 μm thick sections with 3-dimensional co-localization as seen in FIG. 5.

With a single biopsy, 65% of the patients had cutaneous phosphorylated α-synuclein detected, with a 2nd biopsy 87% had cutaneous phosphorylated α-synuclein detected, and with a 3rd biopsy>95% of the cases had cutaneous phosphorylated α-synuclein detected. The addition of the 4th and 5th biopsies did not change the diagnosis for any of the 45 subjects. With this data we established that 3 biopsies were the optimal number to detect cutaneous phosphorylated α-synuclein. To assess accuracy and precision we conducted comparative studies of test reliability, conducted under blinded conditions, performed between 2 laboratories: a total of 35 synucleinopathy patients, 20 healthy control subjects and 15 disease control subjects had biopsies processed at both laboratories. Each patient had 3 biopsies tested (total 210 biopsies in total).

All slides were reviewed in a blinded fashion by two (2) investigators in two different laboratories and independently reported to determine inter-rater reliability. In cases of disagreement, the investigators reviewed the slides together to resolve the discrepancy. Biopsies were considered ‘positive’ if discrete nerve fibers stained by PGP9.5 also contained phosphorylated α-synuclein with the fibers, confirmed by co-localized using 3-dimensional imaging as reported in FIG. 5. The inter-laboratory concordance between the 210 biopsies was 93.3%, but the inter-laboratory diagnostic concordance between the 70 patients (3 biopsies per patient) was 100%. In addition, repeated inter-reader concordance training sessions occurred in order to achieve high inter-rater reliability. In the first review of 45 blinded slides, there was agreement in 38/45 slides, resulting in an 84% concordance in first review of the same slides. A 6-hour collaborative training session occurred to ensure standardization in review. In 2nd inter-rater review of 38 slides there was agreement in 35, resulting in a 92% agreement between raters. A 2nd collaborative training session occurred. In a 3rd inter-rater review of slides 54 slides were reviewed, with agreement in 53 slides, resulting in 98% concordance. Tissue sections were also processed in two different laboratories to determine precision of testing (see Table 4). In addition, because skin biopsies are taken at individual testing sites but processed in a central location, there is always the possibility of delays due to shipping disruptions. Therefore, we have tested the stability of phosphorylated α-synuclein immunostaining through repeated stress measures of the skin biopsies including 1) prolonged fixation times (192 hours), 2) repeated freeze/thaw cycles (4 cycles), 3) prolonged heat exposure, 4) repeated shipping across the United states (4 flights back and forth).

In all cases, there was no loss of phosphorylated α-synuclein immunostaining. There was a significant decrease in PGP9.5 immunostaining of epidermal nerve fibers with heat and prolonged fixation, but deeper dermal structures were minimally affected. To assess sensitivity and specificity we tested 50 individuals with PD, 19 individuals with DLB, 21 individuals with MSA and 17 individuals with PAF. In addition, we have studied 62 healthy control subjects and 35 non-synucleinopathy diseases. The individuals with ‘synucleinopathies’ met a clinical diagnosis of PD83, DLB98, MSA84 or PAF31, 99 based on expert consensus criteria. The non-synucleinopathy disease controls had neuropathy (N=16), essential tremor (N=11) and stroke related movement disorder (N=8). All subjects had 3 mm punch skin biopsies taken from the distal leg, the proximal thigh and the posterior cervical region. A summary of the data from the study subjects is provided in Table 5. We conducted a detailed review of the three individuals clinically diagnosed with synucleinopathy (2 with presumed PD and 1 with presumed PAF) that had no evidence of phosphorylated α-synuclein on their skin biopsies. The phosphorylated α-synuclein negative patients with PD had positive DaTscans but were later believed to carry a diagnosis of PSP (a non-synucleinopathy, tauopathy neurodegenerative disease). The individual with presumed PAF who did not have phosphorylated α-synuclein on skin biopsy was found to have antibodies targeting the ganglionic acetylcholine receptor of the autonomic ganglia, consistent with a diagnosis of autoimmune autonomic ganglionopathy (also not a synucleinopathy) and a disease that is treatable with immune modulating therapy. A summary of the data for review of accuracy, precision, sensitivity, specificity is shown in Table 4.

TABLE 4

| Results of testing per biopsy, | | |
|---|---|---|
| and per patient (each patient | | |
| has 3 biopsies) from subjects | | |
| indicated in Table 3. | Results | |
| [symbol] Accuracy per biopsy | 62.5% | (165/264) |
| (Phosphorylated synuclein | | |
| detected in each individual | | |
| biopsy in those with synucleinopathy) | | |
| Accuracy per patient | 97.7% | (86/88) |
| (Phosphorylated synuclein | | |
| detected in at least 1 | | |
| biopsy in those with synucleinopathy | | |
| who have 3 biopsies [symbol] tested) | | |
| Precision: Intra-laboratory | 92.7% | (257/285) |
| testing per biopsy | | |
| (120 synuclein, [symbol] 165 control) | | |
| Precision: Intra-laboratory | 100% | (95/95) |
| testing per patient | | |
| (40 synuclein, 55 [symbol] control) | | |
| Precision: Inter-laboratory | 92.3% | (194/210) |
| testing per biopsy | | |
| (105 synuclein, 105 [symbol] control) | | |
| Precision: Inter-laboratory | 100% | (70/70) |
| testing per patient | | |
| (35 synuclein, 35 [symbol] control) | | |

| | |
|---|---|
| Sensitivity | 97.73% |
| [symbol] Specificity | 100% |
| Positive predictive value | 100% |
| [symbol] Negative predictive value | 97.26% |
| Overall diagnostic accuracy | 98.74% |

[symbol]

indicates data missing or illegible when filed

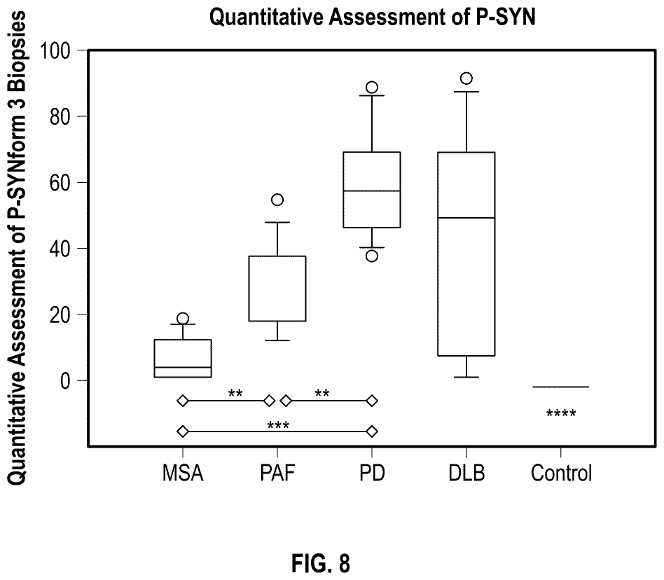

To quantitate phosphorylated α-synuclein, unbiased stereologic quantitation of phosphorylated α-synuclein was performed in 15 subjects each with PD, MSA, PAF and DLB using our previously published techniques. The results, normalized by percentage synuclein to volume of tissue analyzed, range from 0 (no synuclein present) to 100 (maximum distribution of synuclein). The results (FIG. 8) demonstrate that MSA has the lowest peripheral synuclein deposition, followed by PD and DLB with the highest (P<0.001 ANOVA). PAF had overlap with all groups, as anticipated given the high conversion of PAF to PD, MSA and DLB.31

TABLE 5

| | | | | | P-SYN | P-SYN |
|---|---|---|---|---|---|---|
| | Sex | Age | Hoehn & | | positive | positive |
| Disease | (M/F) | (±SD) | Yahr stage | MOCA | (#) | (%) |
| PD | 26/25 | 64 ± 6.3 | 2.0 ± 0.5 | 24.8 ± 4.2 | 48/50 | 96% |
| MSA | 11/10 | 57 ± 4.7 | 2.9 ± 0.6 | 26.0 ± 3.6 | 21/21 | 100% |
| DLB | 9/10 | 67 ± 7.2 | 1.9 ± 0.4 | 20.4 ± 3.1 | 19/19 | 100% |
| PAF | 9/9 | 66 ± 6.8 | 0.5 ± 0.4 | 26.2 ± 2.8 | 17/18 | 94.4% |
| Healthy Control | 31/30 | 58 ± 10.1 | 0.0 ± 0.0 | 26.2 ± 2.4 | 0/61 | 0% |
| Disease Control | 18/17 | 62 ± 8.9 | 1.2 ± 0.8 | 25.9 ± 2.3 | 0/35 | 0% |

Incorporating the quantitative phosphorylated α-synuclein into a ROC curve analysis using thresholds of 17.3 (PSYN %/MM3) and 44.6 (PSYN %/MM3) differentiates between synucleinopathy subtypes with ˜82% sensitivity and specificity. The inclusion of demographics, vital signs and clinical examination data into an algorithm that includes quantitative phosphorylated α-synuclein improves the ability to differentiate between the synucleinopathies to ˜87% sensitivity and specificity.

### Example 4

A cross sectional study of individuals with synucleinopathy, non-synucleinopathy neurodegenerative disease and healthy controls.

The study is designed to assess the following specific aims:

Specific Aim 1: To define the test accuracy and precision of skin biopsy detection of phosphorylated α-synuclein.

Specific Aim 2: To define the sensitivity and specificity of skin biopsy detection of phosphorylated α-synuclein deposition for the diagnosis of synucleinopathies.

Specific Aim 3: To differentiate between the synucleinopathies by quantitative measurement of phosphorylated α-synuclein with skin biopsies

Trial Synopsis: In collaboration with many hospital systems and movement disorder centers we will recruit patients with PD, MSA, DLB and PAF into the study. A total of 300 subjects with synucleinopathy will be included. A total of 100 non-synucleinopathy subjects will be recruited into the study comprised of healthy control subjects. In addition, biopsies from 100 healthy control subjects from a parallel study conducted by Cutaneous NeuroDiagnostics will be included in this analysis for a total of 200 control subjects (20 of each sex by decile from 40-49, 50-59, 60-69, 70-79 and 80-99 (only 20 of each gender to be included in the 80-99 year age group due to more difficulty in finding ‘healthy’ subjects at that age range). Routine skin biopsies will be performed at three sites on each patient. Detailed quantified examination, history and ancillary testing will be provided to allow confirmation of synucleinopathy diagnosis by two disease experts. Biopsies will be shipped to Cutaneous NeuroDiagnostics for central processing.

Approach: This is a prospective cross-sectional study to acquire tissue for blinded analysis of phosphorylated α-synuclein. The skin biopsies will be used to define the accuracy, precision, sensitivity and specificity of diagnosis using skin biopsy in individuals with clinically confirmed synucleinopathies and comparing the results to non-synucleinopathy healthy and disease controls. Quantitation of phosphorylated α-synuclein will be used to differentiate between synucleinopathies. A total of 500 participants (300 with synucleinopathy and 200 without synucleinopathy) will be included. We estimate the breakdown of the 300 study participants with synucleinopathy to be 105 with PD, 40 with MSA, 95 with DLB and 60 with PAF. The non-synucleinopathy controls will be comprised of healthy individuals (without evidence of neurodegenerative disease) across a range of ages from 40-99 (20 per sex, per decile, (with 20 for age 80-99)) for a total of 200 subjects (100 recruited during this study and 100 controls included from a parallel study).

Study visits will include 1) review of inclusion and exclusion criteria 2) signing the informed consent 3) review of medical history and current medications 4) quantified neurological examinations (MDS-UPDRS/UMSARS)67, 71 and Hoehn and Yahr scores100 5) cognitive testing (MOCA, Trails), 6) quality of life and disease questionnaires 7) Orthostatic vital signs and 8) skin punch biopsies (3-mm) taken from the distal leg, proximal thigh and posterior cervical region using 1% lidocaine for anesthesia.

Quantified neurologic examinations. All participants will have standardized neurologic examinations using published methods. The specific examinations will be keyed to the clinical diagnosis and will include MDS-UPDRS 71 for subjects with presumed PD and DLB, and UMSARS for subjects with a clinical diagnosis of MSA or PAF67. All subjects will have Hoehn and Yahr scores. The neurological examinations will be performed by the treating physicians at the various study enrollment centers. Orthostatic vital signs will be measured in all study subjects.

Consensus diagnosis of synucleinopathy: The physicians who examine the subject will indicate the presumed diagnosis (PD, MSA, DLB, PAF). The subject history, examination scores, medical records and ancillary test data will then be sent for central review by 2 disease experts to confirm the subject's diagnosis of synucleinopathy (PD, MSA, DLB or PAF). In the setting of a disagreement, a 3rd disease expert will adjudicate the final decision for diagnosis. If, in the consensus panel's opinion, a subject is unlikely to have a synucleinopathy, they will be excluded from the study. The consensus panel will make the final decision on which category of synucleinopathy a potential study subject is ultimately placed into.

Questionnaires: All participants will complete the following questionnaires: the European Quality of Life Instrument (EQ-5D and EQ-VAS), Parkinson's Disease Questionnaire-39 (PDQ-39), the orthostatic hypotension questionnaire (OHQ)108 and the REM sleep behavior disorder screening questionnaire.

Skin biopsies: Skin biopsies will be obtained using standard protocols, with 3-millimeter punch biopsies taken from the distal leg, the distal thigh and the posterior cervical region after local anesthesia with 1% lidocaine. The routine acquisition of 3 skin biopsies for small fiber neuropathy testing has provided a clinical standard for this approach and it is approved by patients. These small biopsies do not require sutures and are simply covered with a Band-Aid. Wound care instructions are provided.

Skin biopsy processing and immunostaining: The skin samples with be placed in a labeled tube with Zamboni fixative and shipped over night to a lab using a standardized biopsy kit. When received, the samples will be washed and placed in a cryoprotectant. The skin samples will then be sectioned into 50 micrometer thick samples using a cryostat. Tissue is immunostained with free-floating sections using our standard protocols. A total of 8 tissue sections per biopsy will be analyzed. The slides are incubated with anti-mouse phosphorylated alpha-synuclein s-129, followed by incubation in biotin conjugated anti mouse for 2 hours. This is followed by incubation with streptavidin conjugated fluorescence dye Cy3. Sections are washed, followed by incubation with rabbit anti-PGP9.5 overnight. Sections are then washed and conjugated with anti-rabbit fluorescence dye Cy2 for 2 hours. After immunostaining, sections are mounted using aqueous mounting medium. In all experiments, positive and negative controls are run to confirm results.

Skin biopsy imaging and analysis: All biopsies will be evaluated in a blinded fashion by two independent investigators using standard protocols. Slides will be blinded and batched for review to prevent unblinding. A total of 1500 biopsies from 500 study subjects will be reviewed in this blinded fashion. All skin biopsies will have quantitation of nociceptive C-fiber density IENFD in fibers per millimeter of tissue, and quantitation of phosphorylated α-synuclein.

Quantitation of phosphorylated α-synuclein: Unbiased stereologic quantitation of phosphorylated α-synuclein will be measured incorporating our previously published methodology. Images will have a grid mask overlay on top of the image using a cycloid test system. Briefly, a grid of curved cycloids is placed over both the phosphorylated α-synuclein and PGP9.5 florescent images, and the intersection points between the cycloids and nerve are noted using a 3-dimensional stepping pattern using Image Pro-Plus (Media Cybernetics). Regions of cycloid co-localization with both PGP9.5 and phosphorylated α-synuclein with fractionator sampling will estimate the total length of phosphorylated α-synuclein within nerve fibers co-localized with PGP9.5 with results expressed as PSYN %/MM3. Results of the quantitative phosphorylated α-synuclein analysis and the results of the intra-epidermal nerve fiber density analysis will be incorporated into the analysis of the synuclein subgroup specific aim.

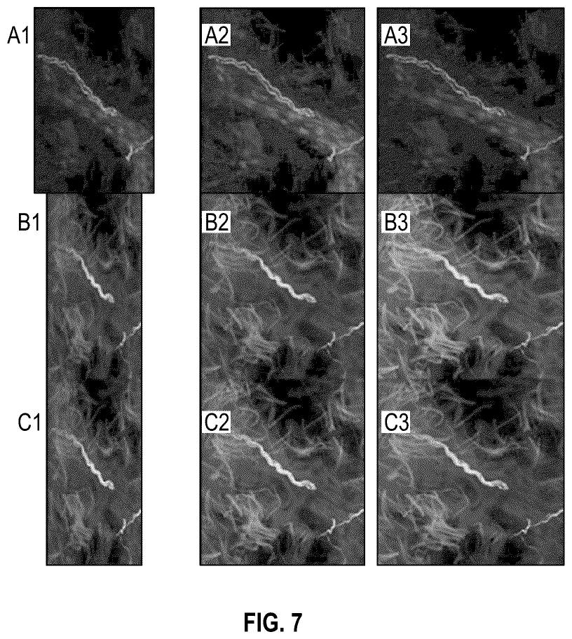

Definition of ‘positive biopsy results’: Only biopsies expressing phosphorylated α-synuclein that co-localizes within a PGP9.5 immunostained sudomotor, vasomotor or pilomotor nerve fiber will be considered positive. Any equivocal section will be considered negative. All positive sections will undergo confocal Z-stack imaging with 3D colocalization for confirmation of results as noted in FIG. 7.

Automated image analysis: As an exploratory endpoint, skin biopsy images will undergo automated image analysis using artificial intelligence (AI) algorithms to define positive results using unbiased thresholding. We will explore automated image analysis techniques using Image Pro-Plus (Media Cybernetics) and Neurolucida 360 (MBF Bioscience). Phosphorylated α-synuclein within nerve fibers will be detected using U0Net convolutional neural networks, which aims to achieve full-image segmentation by determining a pixel-wise segmentation map. The models will be developed using Python 3.5.2, Tensorflor 1.0.0 and Keras 1.2.1 and trained on 200 iterations initially from previously obtained images. Test images from 1500 biopsies will be included as the final data set, with final pathological review by 2 readers as the gold standard. Automated AI image analysis will continue in an iterative fashion to optimize future automation of results.

Recruitment: Participants in the study will be recruited from a combination of academic centers and smaller specialty practices across the U.S.

Follow up: A yearly phone call follow up to all recruiting centers to quickly review if there has been any change in diagnosis of any of the patients they have referred into the study. This information will be included in post-study follow up to aid in understanding the implications of the study results over time.

Data management: All study data will be recorded electronically into a REDcap database. All data will be secured behind a firewall.

Study blinding: All investigators reviewing slides will be blinded to subject data. The consensus panel investigators will review data online and vote electronically for final decisions and will be blinded to biopsy results. If there is a disagreement on diagnosis between the two consensus reviewers, the case will be adjudicated by a 3rd expert.

Expected Outcomes: We anticipate, based on our extensive preliminary data, that: 1.) Individuals with a synucleinopathy (PD, DLB, MSA or PD) will have high rates of phosphorylated α-synuclein present on skin biopsy (>90% accuracy for all synucleinopathies). 2.) We also expect that healthy control subject, across ages and genders, will have no evidence of phosphorylated α-synuclein present within their skin biopsies (100% specificity); 3) We expect that our results will be congruent between repeated tests at the same laboratory, and between repeated tests at different laboratories, 4) We anticipate that inter and intra-rater reliability will be >95%, 5) We also expect, based on our preliminary data, that the use of 3 skin biopsies per subject will provide the optimal ROC curve characteristics for maintaining high sensitivity and specificity while avoiding overly excessive numbers of skin biopsies to confirm a diagnosis. Finally, we anticipate that quantitation of phosphorylated α-synuclein deposition will provide the ability to differentiate between synucleinopathy subtypes.

Exploratory Endpoints: An assessment of artificial intelligence supplemented image analysis with unbiased thresholding as a method for detecting phosphorylated α-synuclein.

Statistical Analysis: For specific aims 1&2, the outcome of the presence or absence of phosphorylated alpha-synuclein will be dichotomous. The primary endpoint for SA1 is accuracy and precision. The performance of phosphorylated α-synuclein will be measured against the accuracy of diagnoses as determined by the consensus panel diagnosis of synucleinopathy. Accuracy will be determined by synucleinopathy subtype and by total diagnosis of synucleinopathy. Final test accuracy will be calculated by true positive and true negative tests divided by all tests (synucleinopathy (N=300) and healthy control subjects (N=200)). Precision will be determined by interclass correlation coefficients, testing results between samples run at the same laboratory, and testing between different laboratories. Inter and intra-rater reliability will be measured across testing with interclass correlation coefficients. For SA2: Sensitivity and specificity will be calculated using standard measures. For SA3: calculation of receiver operating characteristics (ROC) curves will compare quantitative analysis of phosphorylated α-synuclein and synucleinopathy subtype. Inclusion of demographic information, vital signs and clinical information and quantitative phosphorylated α-synuclein into multivariate logistic regression model to differentiate between synucleinopathy subtypes. Additional secondary analysis will include reporting of data from disease control subject using descriptive statistics that will include questionnaire and examination scoring data by biopsy result. Bland-Altman plots will be used to test quantitation of synuclein deposition for both accuracy and precision testing.

Exploratory endpoints include will include exploratory factor analysis to identify complex relationships including location of phosphorylated α-synuclein deposition within skin biopsies, consensus panel diagnosis, examination scores, questionnaire results and demographic variables to determine if natural groupings occur. Additional exploratory endpoints will include image analysis using convolutional neural networks to allow for construction and extraction of flexible representative features from input data and evaluate multiple layers of extracted features. Iterative analysis of results will be compared to final diagnosis by pathology.

Power Analysis. To determine if phosphorylated α-synuclein has the minimally acceptable test performance characteristics to differentiate synucleinopathy from non-synucleinopathy, we will define the true positive fraction at 0.90 and the false positive fraction at 0.01.112 With a two sided alpha of 0.01 and a power of 0.9, we estimate that a total of 150 patients with synucleinopathy and control are required to complete this study. In order to validate testing on and between subtypes of synucleinopathy (PD, MSA, DLB, PAF), we have a total of 300 subjects with synucleinopathy. The 200 healthy control subjects are required to provide adequate numbers for normative data across sex and age. Based on preliminary data, phosphorylated α-synuclein will not be detected in healthy subjects. Individuals with anosmia, constipation or REM sleep behavioral disorder are excluded from the control group. We do anticipate that a very small number (up to 1.5%) of control subjects might have synucleinopathy in the 200 healthy controls.

### Example 5

### Single Laboratory Validation of Cutaneous Phosphorylated Alpha-Synuclein Detection.

Background: We have previously reported that phosphorylated α-synuclein (P-SYN) can be detected within cutaneous nerve fibers of patients with synucleinopathies with high sensitivity and specificity. We now report the large scale validation of P-SYN detection within a central laboratory.

Objective: To validate testing of cutaneous P-SYN detection within a single centralized laboratory.

Methods: Seventy patients with a clinical diagnosis of synucleinopathy (20 PD, 15 MSA, 20 PAF and 15 DLB) previously confirmed by cutaneous P-SYN on skin biopsy and 40 healthy control subjects were included. All subjects had 3 skin biopsies taken from the distal leg, distal thigh and posterior cervical region with biopsies shipped to a central laboratory for immunostaining with PGP9.5 and P-SYN. Each skin biopsy was immunostained 3 separate times over 6 months. Accuracy, sensitivity, specificity are calculated with repeatability measured for each case, and each biopsy.

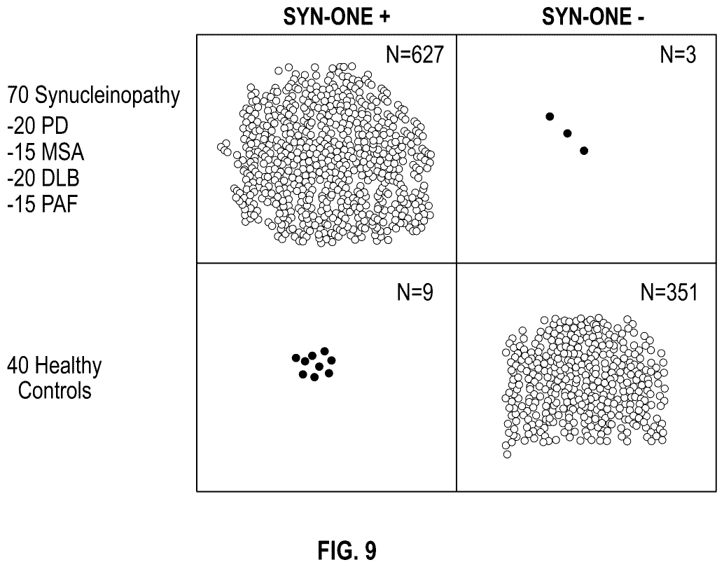

Results: As illustrated in FIG. 9, 110 individuals had a total of 330 biopsies evaluated 3 times, for 990 measurements. Accuracy of P-SYN detection was 99.1%, Sensitivity was 99.7%, Repeatability per case (with 3 biopsies) was 100%, per biopsy repeatability was 99.8%. Specificity was 97.5% with 1 control case repeatedly testing positive on all 3 biopsy sites. Follow up clinical evaluation and polysomnography confirmed REM sleep behavioral disorder in the positive control case.

Discussion: The test-retest accuracy, sensitivity, specificity and repeatability are high using cutaneous P-SYN detection with a central testing laboratory. A single control case contained P-SYN on all biopsies and in all repeated measurements and was later determined to have a synucleinopathy (REM sleep behavioral disorder). This validation study provides support for use of cutaneous P-SYN detection in the diagnosis of synucleinopathies,

### Example 6

### Cutaneous Alpha-Synuclein Deposition in Postural Tachycardia Patients (POTS).

Objective: To report a case series of patients with neuropathic POTS and cutaneous phosphorylated alpha-synuclein (P-SYN) deposition on skin biopsy and compare these to neuropathic POTS patients without P-SYN deposition.

Methods: The medical history, physical examination findings, autonomic function testing and skin biopsy neuropathology of patients under the age of 50 with a postural tachycardia and a diagnosis of POTS were retrospectively reviewed. Included patients completed the composite autonomic severity score (COMPASS 31), the Wood Mental Fatigue Inventory, the Epworth Sleepiness scale, the REM Behavior Disorder Questionnaire, the Patient-reported Outcomes Measurement Information System (PROMIS-10), and the Gastroparesis Cardinal Symptom Index.

The results are reported by descriptive statistics, with mean±standard deviation reported unless otherwise noted. Results were categorically analyzed by the presence, or absence, of phosphorylated alpha-synuclein on skin biopsy, with results compared by Fishers exact test, unpaired t-test, or Kruskal Wallis testing if results were not normally distributed. Pearson correlations were used to describe relationships between tests. A P value of <0.05 was considered significant. Bonferroni corrections were made for multiple comparisons. Statistical analysis completed by SPSS 20 (SPSS, IBM Inc).

TABLE 6

| | P-SYN | P-SYN | P |
|---|---|---|---|
| Demographic Detail | Positive | Negative | values |
| Number | 7 | 1 | |
| Age | 31.1 (22-47) | 32.7 (19-46) | NS |
| Sex (% Female) | 43% | 93% | <0.05* |
| Duration of Symptoms (years) | 6.5 ± 5.1 | 6.4 ± 5.9 | NS |
| Mean number of Comorbid | 0.4 | 0.4 | NS* |
| Medical Conditions | | | |
| Family history of synucleinopathy | 2/7 | 1/15 | NS* |
| History of thermal dysregulation | 1/7 | 4/15 | NS* |
| History of resting tremors | 0/7 | 3/15 | NS* |
| History of anosmia | 0/7 | 1/15 | NS* |
| History of confusion or | 0/7 | 8/15 | <0.05* |
| hallucinations | | | |
| History of a sleep disorder | 5/7 | 0/15 | <0.001* |
| History of gastroparesis | 5/7 | 0/15 | <0.001* |
| History of constipation | 6/7 | 4/15 | NS* |
| History of bladder dysfunction | 2/7 | 4/15 | NS* |

Results: Of 296 patients seen with POTS, 22 patients with suspected neuropathic POTS had skin biopsies performed during their evaluation. Seven of 22 patients had P-SYN present on skin biopsy, while 15 individuals did not. Those with P-SYN on biopsy were: (1) more likely to be male; (2) had features of REM sleep behavioral disorder; (3) reported less sleepiness and cognitive impairment; and (4) noted greater symptoms of gastroparesis. On autonomic testing, the group with P-SYN deposition was more likely to have a hypertensive response to tilt-table testing and abnormal QSART responses.

TABLE 7

| | P-SYN | P-SYN | |
|---|---|---|---|
| Test | Positive | Negative | P values |
| Heart Rate Deep Breathing | 1.29 ± 0.13 | 1.3 ± 0.12 | NS |
| (ratio) | | | |
| Valsalva Ratio | 2.37 ± 0.44 | 2.01 ± 0.49 | NS |
| Baseline Heart Rate | 72 ± 13 | 78 ± 8 | NS |
| (beats per minute) | | | |
| Highest heart rate during | 106 ± 14 | 111 ± 9 | NS |
| 10 minute tilt | | | |
| Change in heart rate | 33.7 ± 4.3 | 31.7 ± 3.9 | NS |
| during tilt | | | |
| Baseline SBP (mmHg) | 121 ± 13 | 117 ± 12 | NS |
| Highest SBP during tilt | 153 ± 23 | 125 ± 14 | <0.01 |
| (mmHg) | | | |
| Delta SBP change during tilt | 32 ± 17 | 9 ± 8 | <0.01 |
| (mmHg) | | | |
| Baseline DBP(mmHg) | 74 ± 7 | 71 ± 7 | NS |
| Delta DBP change during tilt | 23 ± 13 | 15 ± 8 | NS |
| (mmHg) | | | |
| Highest DBP during tilt | 97 ± 15 | 86 ± 11 | NS |
| (mmHg) | | | |
| QSART abnormal | 86% | 13% | <0.05* |
| (percent of patients) | | | |
| IENFD distal leg | 11.4 ± 3.6 | 11.4 ± 4.6 | NS |
| IENFD distal thigh | 15.8 ± 4.4 | 17.5 ± 3.7 | NS |
| IENFD posterior cervical | 30.0 ± 3.7 | 29.9 ± 6.1 | NS |

Conclusion: Phosphorylated alpha-synuclein deposition is present in some postural tachycardia patients with neuropathic features. Individuals with a postural tachycardia and cutaneous phosphorylated alpha-synuclein deposition may be distinguished from other patients with neuropathic POTS.

### Example 7

Lubag is an X-Linked parkinsonian disorder with significant dystonia present.

The traditional detection of neurodegenerative diseases relies on the clinical observation of detectable neurologic changes followed by ancillary testing such as imaging, serologic evaluation, and neuropsychological testing. Unfortunately, as these diseases are believed to be due to the accumulation of proteins over years or decades, by the time these clinical changes are observed the ability to slow or reverse the destructive process is possibly too far gone. Current pharmacologic strategies would be significantly improved if one could detect these biologic changes much earlier in the disease process. This would require the development of a very sensitive test with very high specificity. With such a test the treatment of pre-symptomatic individuals would be a reality. We have determined that the Syn-One test has achieved these milestones.

Data generated using methods and systems described herein has shown in several disease states the ability to detect phosphorylates alpha-synuclein in skin biopsies of patients long before others thought possible.

One synucleinopathy is pure autonomic failure. These patients typically present in their 5^{th} or 6^{th} decade. They develop signs of being unable to regulate their heart rate, blood pressure, gastro-intestinal function, and temperature regulation among others. The diagnostic test of choice is autonomic reflex screens. Sadly these patients have a very high probability of continuing to develop into other synuclein disorders such as Parkinson's disease and Multiple System atrophy indicating that these diseases cause by accumulation of synuclein are along a continuum. Using the Syn-One test we have been able to identify patients who are in their second and third decade, who do not meet the criteria for pure autonomic failure by autonomic testing, but who have some autonomic symptoms. These patients have been found to have phosphorylated synuclein within their skin biopsies suggesting they may be earlier along the continuum but still part of the same synuclein pathology. This data now in press indicates that the Syn-One test has shown the earliest ability of any published test to detect abnormal synuclein deposition.

As a second example, the Syn-One test has been proven to be able to detect previously unknown disorders of synuclein deposition. Lubag is an X-linked parkinsonian disorder with significant dystonia present. The genetic basis of the disease is linked to the TAF1 gene on chromosome Xq13.1 and it is much more common is people of Phillipino descent. Although this disorder has links to Parkinson's disease no one had studied the possibility of Lubag being a synucleinopathy. This was because it is a rare disease and having a sensitive effective method for detecting synuclein was not available. One of our collaborators had a small collection of patients with Lubag and using the Syn-One test was the first to demonstrate that patients with Lubag do in fact have a Synucleinopathy (this is submitted).

These two case studies are just the first of many. Being able to detect phosphorylated synuclein deposition before any other test provides enormous possibilities for earlier pharmacologic intervention. And being able to simply and reliably test many disorders will allow better characterization of the number of disorders truly linked to synuclein deposition.

### Example 8

Diagnostic test that distinguishes DLB from AD and provides quantitative metrics for DLB severity.

These studies will have an immediate impact on the clinician's ability to accurately distinguish DLB from AD during routine examinations and develop appropriate disease management strategies for each patent. The long-term impact of this work lies in defining the quantitative metrics of DLB progression, which can serve as measures of target engagement and surrogate endpoints in clinical trials, thereby accelerating development of therapies for Lewy body disorders.

Many of the clinical features of DLB, including problems with memory and reasoning, overlap with AD patients, resulting in over 25% of DLB patients being misdiagnosed with AD. Historically, for DLB, the only way to visualize the abnormal synuclein protein accumulation was through brain biopsies or at autopsy and therefore was never available to patients or their treating physicians. The ability to detect phosphorylated α-synuclein through a skin punch biopsy will alter the management of patients with DLB by providing improved diagnostic accuracy and an efficient testing modality within the context of the healthcare system as a whole. The use of skin biopsies to study nerve related disorders has been used commercially for over a decade.

The development of a more accurate and easily accessible test for DLB would save patients from countless unnecessary tests and prevent exposure to ineffective medications, while also identifying patients early in the clinical course for more timely intervention. Doctors would also benefit from the use of the Syn-One Test to accurately diagnose DLB patients. Currently, the diagnosis of DLB and AD is primarily based on clinical criteria. Currently, diagnostic accuracy may only be 30% as the clinical manifestations are largely overlapping between DLB and AD. A fast, reliable, and easily accessible diagnostic as the Syn-One Test will remove a huge burden on doctors. In addition, molecular imaging used for DLB and AD diagnosis are typically available only in academic or large regional medical centers limiting access for many patients. In addition, the specific and sensitive test to diagnose patients with DLB will help to accelerate the development of novel neuroprotective agents. Biomarkers of disease are critically important to pharmaceutical companies because they can shorten the duration of clinical trials, reduce the numbers of patients needed for studies, and increase the chances of success of a novel treatment.

DLB and AD share many similar features and differentiating DLB from AD is of clinical importance because individuals with DLB respond better to cholinesterase inhibitors but are more sensitive to neuroleptics, which may cause clinical decline.

### Example 9

Cutaneous α-Synuclein Signatures in Patients with Multiple System Atrophy and Parkinson Disease.

Multiple system atrophy (MSA) is a progressive neurodegenerative disorder caused by the abnormal accumulation of alpha-synuclein in the nervous system. MSA is traditionally classified as a CNS predominant synucleinopathy. The pathological hallmark of MSA is the presence of misfolded phosphorylated alpha-synuclein (P-SYN) predominantly within glial cytoplasmic inclusions, thus classifying MSA as an oligodendroglial alpha-synucleinopathy. Clinical features of MSA include autonomic and motor dysfunction, which overlap with those of Parkinson disease (PD), particularly at early disease stages. There is an unmet need for accurate diagnostic and prognostic biomarkers for MSA, and, specifically, a critical need to distinguish MSA from the other synucleinopathies, particularly PD. The purpose of the study was to develop a unique cutaneous pathological synuclein signature of phosphorylated alpha-synuclein that could distinguish patients with MSA from patients with PD and healthy controls.

This exemplary study included 31 patients with MSA and 54 patients with PD (both as diagnosed according to current clinical consensus criteria), along with 24 matched controls. All participants underwent neurologic examinations, autonomic testing and skin biopsies at three locations. The density of intra-epidermal, sudomotor and pilomotor nerve fibers was measured. The deposition of phosphorylated alpha-synuclein in the skin biopsy tissues was quantified. Results were compared to clinical rating assessments and autonomic function test results.

Patients with PD had reduced nerve fiber densities compared to patients with MSA (P<0.05, all fiber types). All patients with MSA and 51/54 with PD had evidence of phosphorylated alpha-synuclein in at least one skin biopsy. No phosphorylated alpha-synuclein was detected in controls. Patients with MSA had greater phosphorylated alpha-synuclein deposition (P<0.0001) and more widespread peripheral distribution (P<0.0001) than patients with PD. These results provided >90% sensitivity and specificity in distinguishing between the two disorders.

Alpha-synuclein is present in peripheral autonomic nerves of MSA patients, and when combined with synuclein distribution, accurately distinguishes MSA from PD. This example illustrates various embodiments of systems and methods of measuring phosphorylated alpha-synuclein in skin biopsies from a group of patients, and differentiating a set of patients with MSA from a set of patients with PD.

### Study Group

A prospective single visit cohort study has three different groups: 1) individuals with MSA, 2) PD and 3) healthy controls. Patients with MSA and PD were recruited by the autonomic and movement disorders groups at the Neurology Departments of the Beth Israel Deaconess Medical Center (BIDMC), New York University (NYU) and University of Colorado were recruited for study participation from 2016-2021.

Healthy control subjects were recruited by local advertisement. Patients with MSA were included if they met criteria for ‘probable’ MSA using current diagnostic criteria for MSA. Patients with PD were included if they met criteria for ‘definite PD’ using the UKPDS brain bank criteria with 3 or more supportive features including diagnosis>5 years and excellent levodopa response. Exclusion criteria for MSA and PD followed diagnostic guidelines. Other exclusion criteria included anticoagulation use, any underlying disorder that could cause neuropathy or risks of complication to a skin biopsy (e.g., allergy to lidocaine). Controls were included if they had no known medical conditions. Exclusion criteria for healthy controls included family history of synucleinopathy, clinical features of neuropathy or history suggestive of rapid eye movement sleep behavior disorder (RBD).

All participants underwent a general history and detailed neurological examination. MSA disease severity was rated by the Unified Multiple System Atrophy Rating Scale (UMSARS), while PD severity was rated by the Movement Disorders Society Unified Parkinson's Disease Rating Scale (MDS-UPDRS). Both disorders were also rated with the Hoehn and Yahr scale. Most participants completed the University of Pennsylvania Smell Identification test (UPSIT), and all completed the Montreal cognitive assessment (MOCA).18 All participants completed orthostatic blood pressures in the supine resting position and after 3 minutes of standing. All testing and examinations were completed in the OFF state for medications used to treat motor symptoms (e.g., levodopa, dopaminergic agonists) and for the symptomatic treatment of orthostatic hypotension (e.g., midodrine, droxidopa). Participants completed the orthostatic hypotension questionnaire (OHQ), the single question REM sleep behavioral disorder questionnaire, the MSA red flags questionnaire and the Parkinson's disease questionnaire.

Cardiovascular parasympathetic function testing (the heart rate response to deep respiration and the Valsalva maneuver) and cardiovascular sympathetic function (the blood pressure response to a Valsalva maneuver and tilt-table testing to 60 degrees for 10 minutes) was performed in patients from BIDMC and NYU. Continuous ECG monitoring, continuous beat-to-beat blood pressure recordings and manual blood pressure measurements every minute during tilt-table testing were recorded.

### Pathological Analysis

Three-millimeter punch skin biopsies were obtained from the lateral distal thigh (20 cm above the lateral knee), proximal thigh (20 cm below the lateral hip), and posterior cervical region (2 cm lateral to the C7 spinous process) after local anesthesia with 2% lidocaine. Skin biopsy specimens were fixed in Zamboni solution for 18 hours and cryoprotected overnight (20% glycerol and 20% 0.4 M Sorreson buffer). Tissue blocks were cut by freezing microtome into 50 μm thick sections. An average of 12 tissue sections were analyzed per biopsy with different immunostaining combinations. Additional sections were analyzed in cases where sweat glands or arrector pili muscles were not identified within the original sections. Pathological evaluation of specimens was completed.

Double labeled immunofluorescent staining was performed using our previously published methods for covisualizing total nerve fibers and alpha-synuclein positive nerve fibers. The primary antibodies used to visualize total nerve fibers included the pan-axonal marker protein gene product 9.5 (PGP 9.5, rabbit polyclonal, UltraClone, RA95101; 1:10,000 or PGP 9.5, mouse monoclonal, RA9503; Ultraclone; 1:1000), the polyclonal antibody targeting total alpha-synuclein recognized multiple binding sites from amino acids (rabbit polyclonal, AB5038P, Chemicon 1:13,000) and antibody targeting phosphorylated alpha-synuclein (mouse monoclonal, Covance, New Jersey, MMS-5091,1:500). Dual immunostaining with PGP 9.5 and phosphorylated or PGP 9.5 and total alpha-synuclein was performed for all samples as previously described herein and in the literature.

All the stained sections were initially examined under a fluorescent microscope, with areas of interest imaged by confocal microscopy (Zeiss LSM5 Pascal Exciter; Carl Zeiss, Thornwood, NY). A series of images of optical sections were acquired at 3 m intervals throughout the depth of the 50 μm section as a z-stack (Carl Zeiss, Lens Plan-Apochromat ×20/0.8). The confocal scan condition was set based on optimal ratio of signal intensity to noise for the nerve fibers stained with different primary antibodies of slides from control subjects, then the confocal parameters for scanning were fixed in all slides scanned.

Intra-epidermal nerve fiber density (IENFD): Biopsies underwent blinded IENFD counting with PGP9.5 positive fibers and results were expressed as number of fibers crossing the dermal-epidermal junction per millimeter. Sweat gland nerve fiber density (SGNFD): Sweat glands were imaged and nerve fiber densities quantified. Briefly, 30 μm deep Z-stack composite confocal images were created for each sweat gland and the gland was highlighted as the area of interest. PGP9.5 innervated fibers were detected within the area of interest and overlapping regions of P-SYN were quantified as the proportion of total PGP9.5 innervated fibers. The results are expressed as a ratio of the P-SYN to PGP9.5 innervated fibers. Sweat gland innervation was evaluated in a blind fashion.

Pilomotor nerve fiber density (PMNFD): Pilomotor muscles were imaged, and nerve fiber densities were quantified. The average number of nerve fibers intersecting five horizontal lines across the width of the pilomotor muscle in 3 μm thick confocal images were reported in fiber/mm. The PMNFD was reported for PGP 9.5 and alpha-synuclein.

Phosphorylated alpha-synuclein quantitation: Slides were deidentified and all slide readers were blinded to study subject diagnosis. Phosphorylated alpha-synuclein positive fibers were required to be completely co-localized within PGP9.5 positive nerve fibers by review of Z-stack image analysis (i.e. areas of synuclein that did not co-localize were defined as artifact. The number of positive results was noted for each cutaneous dermal structure for each tissue section for each biopsy. For example, if one or more synuclein positive fibers was found to innervate a single pilomotor muscle, the pilomotor muscle is defined as ‘one’ synuclein positive structure. A second, separate pilomotor muscle with one or more synuclein positive fibers would count as a 2nd P-SYN positive structure. A quantitative analysis of phosphorylated alpha-synuclein was performed for each patient. Results were expressed as the number of P-SYN positive structures. The location of P-SYN deposition was calculated across the various biopsies using a distribution coefficient at the posterior cervical, distal thigh and distal leg.

The calculation of the distribution coefficient (DC)=(PCscore−DLscore)/(PCscore+DTscore+DLscore) where the posterior cervical (PC), distal thigh (DT) and distal leg (DL) scores are the quantitative analysis of synuclein deposition at each biopsy site. Scores with ‘zero’ in the numerator were assigned a score of zero. The scores ranged from (+)1 (indicating that P-SYN was located only at the posterior cervical site only) to (−)1 (indicating that P-SYN was located at only the distal leg site) and indicated the relative distribution of P-SYN from a proximal to distal gradient.

Statistical data analysis was performed using SPSS v17.0 (IBM, Chicago, IL). Data are presented as mean±standard deviation. Group differences in total nerve fiber density, total alpha-synuclein ratio, phosphorylated alpha-synuclein deposition and autonomic test results are reported. Differences between groups were analyzed by ANOVA with the Holm-Sidak post-hoc test when normally distributed (Shapiro-Wilk). If data were not normally distributed, Kruskal-Wallis with Dunn's method for post-hoc analysis was used. Sensitivity and specificity were calculated for each individual test and in combinations to identify optimal combinations of tests to distinguish PD vs. MSA using receiver operating characteristic (ROC) curves. Cut-off thresholds for the ROC were selected by the highest product of sensitivity*specificity with an edge towards specificity in situations where equivalent results for 2 values were obtained. An exploratory analysis of a combination of ROC curve results was included using an ‘AND’ operator where both results above the preselected ROC threshold were positive, but if either or both were below the ROC threshold the result was negative. Discrimination between groups using ROC curves is reported as the area under the curve (AUC). Associations between alpha-synuclein deposition, autonomic test results and examination scores are expressed as Pearson correlations.

### Results

Thirty-one patients with MSA (15 men, 16 women, mean age 61±9 years, body mass index of 30.7±2.4 kg/m²; 17 had MSA-C and 14 MSA-P), 54 patients with PD (30 men, 24 women; mean age 64±8 years, body mass index of 29.2±2.6 kg/m²) and 24 healthy controls (12 men, 12 women; mean age 62±8 years, body mass index of 31.9±3.1 kg/m²) participated in this study. Table 8 summarizes the participant's demographic information and their disease specific scores.

TABLE 8
Demographic characteristics of participants

| | MSA | PD | Control | P-value |
|---|---|---|---|---|
| | (N = 31) | (N = 54) | (N = 24) | (ANOVA) |
| Sex | 16 F, 15 M | 24 F, 30 M | 12 F, 12 M | 0.47* |
| Age | 61 ± 9 | 64 ± 8 | 62 ± 8 | 0.80 |
| BMI | 29.7 ± 2.6 | 29.2 ± 2.8 | 30.9 ± 2.6 | 0.20 |
| Duration of | 3.4 ± 2.7 | 4.9 ± 4.2^{†} | NA | <0.0001 |
| Disease | | | | |
| Disease- | UMSARS-1: | MDS-UPDRS- | NA | NA |
| specific | 21.8 ± 7.7 | total: 49.0 ± 20.2 | | |
| rating | Range (11-44) | Range (15-90) | | |
| scales | UMSARS-2: | MDS-UPDRS-3: | | |
| | 21.6 ± 8.7 | 33.4 ± 13.8 | | |
| | Range (9-43) | Range (7-68) | | |
| Hoehn and | 2.6 ± 0.9 | 2.1 ± 0.5 | 0 ± 0 | 0.021† |
| Yahr | Range (1-4) | Range (1-4) | | |
| MOCA | 25.7 ± 2.7 | 26.2 ± 2.2 | 25.9 ± 3.4 | 0.42 |
| UPSIT | N = 22 | N = 54 | N = 24 | <0.0001 |
| | 32.9 ± 5.6 | 23.4 ± 5.5 ^{†} | 35.8 ± 2.1 ^{‡} | (KW) |

NS: Not significant.
NA: Not available.
KW: Kruskal-Wallis used instead of ANOVA because data not normally distributed.
*Calculated using a 2 × 3 Fisher exact test.
^{†}P < 0.01 PD vs MSA.
^{‡} P < 0.01 control vs PD.

All participants completed orthostatic vital signs and 22 patients with MSA completed heart rate variability to paced breathing and a Valsalva maneuver. All individuals with PD and control subjects completed testing. Table 9 summarizes the autonomic function testing results. As expected, autonomic abnormalities were more severe in the MSA group.

TABLE 9
Autonomic Function Testing

| | MSA | PD | Control | ANOVA |
|---|---|---|---|---|
| Test | (N = 31) | (N = 54) | (N = 24) | P-value |
| Supine SBP (mmHg) | 152 ± 18 | 136 ± 17* | 127 ± 6^{†‡} | <0.0001 (KW) |
| Supine DBP (mmHg) | 89 ± 7 | 82 ± 5* | 78 ± 3^{†} | <0.0001 (KW) |
| Supine HR (beats/min) | 81 ± 12 | 64 ± 7* | 69 ± 11^{†‡} | <0.0001 (KW) |
| Upright tilt SBP (mmHg) | 117 ± 23 | 120 ± 17 | 122 ± 8^{†‡} | 0.27 |
| Upright tilt DBP (mmHg) | 74 ± 6 | 76 ± 5 | 80 ± 3^{†‡} | <0.0001 (KW) |
| Upright tilt HR (beats/min) | 87 ± 9 | 69 ± 7 | 76 ± 12^{†‡} | <0.0001 (KW) |
| Fall in SBP on tilt (mmHg) | 42 ± 18 | 15 ± 6* | 5 ± 6^{†‡} | <0.0001 (KW) |
| | MSA | PD | Control | |
| | (N = 22) | (N = 54) | (N = 24) | |
| Expiratory to inspiratory ratio | 1.12 ± 0.06 | 1.19 ± 0.11* | 1.32 ± 0.11^{†‡} | <0.0001 |
| Valsalva Ratio | 1.24 ± 0.13 | 1.34 ± 0.19* | 1.43 ± 0.11^{†‡} | <0.0001 |
| Valsalva phase 2 systolic | −55 ± 29 | −42 ± 13* | −36 ± 18^{†} | <0.0001 |
| blood pressure drop (mmHg) | | | | |
| Valsalva phase 4 systolic | 1 ± 9 | 10 ± 10* | 26 ± 11^{†‡} | <0.0001 |
| blood pressure overshoot | | | | |
| (mmHg) | | | | |

SBP: systolic blood pressure.
DBP: diastolic blood pressure.
HR: heart rate.
MSA: multiple system atrophy.
PD: Parkinson's disease.
KW: Kruskal-Wallis used instead of ANOVA because data not normally distributed.
*P < 0.01 PD vs. MSA.
^{†}P < 0.01 control vs MSA.
^{‡}P < 0.01 control vs PD.

TABLE 10
Quantitation of nerve fiber density and synuclein deposition

| | | | | | | 95% CI | |
|---|---|---|---|---|---|---|---|
| | MSA | PD | Control | | | for the | AUC P |
| Test | (N=31) | (N=54) | (N=24) | ANOVA | AUC | AUC | value |
| IENFD distal | 10.2 ± 2.4** | 4.6 ± 2.5 | 12.9 ± 2.1** | P < 0.0001 | 0.916 | 0.872-0.965 | <0.0001 |
| leg | | | | | | | |
| IENFD distal | 14.6 ± 3.2* | 9.4 ± 4.1 | 15.3 ± 5.4* | P < 0.0001 | 0.893 | 0.823-0.963 | <0.0001 |
| thigh | | | | | | | |
| IENFD | 30.8 ± 6.2* | 24.9 ± 5.0 | 27.1 ± 5.9 | P < 0.0001 | 0.821 | 0.733-0.907 | <0.0001 |
| posterior | | | | | | | |
| cervical | | | | | | | |
| PMNFD | 53.4 ± 18.5* | 42.3 ± 13.3 | 55.4 ± 10.9 | P < 0.0001 | 0.594 | 0.467-0.723 | 0.16 |
| distal leg | | | | | | | |
| PMNFD | 62.3 ± 13.4* | 53.1 ± 16.1 | 65.1 ± 14.3 | P < 0.0001 | 0.433 | 0.294-0.570 | 0.36 |
| distal thigh | | | | | | | |
| PMNFD | 78.7 ± 19.3* | 65.4 ± 22.5 | 76.2 ± 16.5 | P = 0.042 | 0.412 | 0.282-0.541 | 0.23 |
| posterior | | | | | | | |
| cervical | | | | | | | |
| SGNFD | 0.11 ± 0.05 | 0.09 ± 0.05 | 0.12 ± 0.04 | P = 0.011 | 0.619 | 0.489-0.750 | 0.0.07 |
| distal leg | | | | | | | |
| SGNFD | 0.14 ± 0.06 | 0.13 ± 0.05 | 0.15 ± 0.06 | P = 0.014 | 0.553 | 0.417-0.689 | 0.44 |
| distal thigh | | | | | | | |
| SGNFD | 0.18 ± 0.07 | 0.17 ± 0.07 | 0.18 ± 0.06 | P = 0.16 | 0.458 | 0.324-0.592 | 0.54 |
| posterior | | | | | | | |
| cervical | | | | | | | |
| P-SYN distal | 4.7 ± 3.6** | 0.4 ± 0.9 | 0 ± 0 | P < 0.0001 | 0.873 | 0.790-0.957 | <0.0001 |
| leg | | | | | | | |
| P-SYN distal | 4.8 ± 3.4** | 0.9 ± 1.2 | 0 ± 0 | P < 0.0001 | 0.839 | 0.739-0.939 | <0.0001 |
| thigh | | | | | | | |
| P-SYN | 5.5 ± 3.6** | 2.0 ± 1.2 | 0 ± 0 | P < 0.0001 | 0.792 | 0.665-0.917 | <0.0001 |
| posterior | | | | | | | |
| cervical | | | | | | | |
| P-SYN Total | 15.0 ± 6.1 | 3.2 ± 1.9 | 0 ± 0 | P < 0.0001 | 0.983 | 0.954-1.01 | <0.0001 |
| P-SYN | 0.07 ± 0.37** | 0.64 ± 0.48 | N/A | N/A | 0.825 | 0740-0.910 | <0.0001 |
| Distribution | | | | | | | |
| coefficient | | | | | | | |

IENFD: intra-epidermal nerve fiber density.
PMNFD: pilomotor nerve fiber density.
SGNFD: sweat gland nerve fiber density.
P-SYN: phosphorylated alpha-synuclein.
N/A not applicable.
*P < 0.05 vs PD with Holm-Sidak post-hoc testing,
**P < 0.01 vs PD;
PD with Holm-Sidak post-hoc testing.
Accuracy of PD vs MSA: A measure of the area under the ROC curve analysis in PD and MSA.
NA: no synuclein detected therefore distribution not calculated.
AUC: Area under the curve from the ROC analysis.
95% CI for the AUC: the 95% confidence interval derived from the ROC curve analysis.

In patients with MSA, intra-epidermal nerve fiber densities, a measure of small sensory afferents, was similar to controls at all biopsy sites. In contrast, patients with PD had lower nerve densities at both the distal leg and distal thigh than patients with MSA and controls (Table 10). Pilomotor nerve fiber densities, a measure of sympathetic innervation of smooth muscle, were similar in MSA and controls at all sites but were reduced in patients with PD at the distal leg and distal thigh sites, as documented in Table 10. Although differences in ANOVA between groups were noted in in sweat gland nerve fiber density, a measure of sympathetic sudomotor cholinergic innervation, there were no differences in post-hoc testing between groups at any biopsy site.

Phosphorylated alpha-synuclein was detected within at least one biopsy in all the patients with MSA, in 94% (51/54) of patients with PD and in none of the control subjects as reported in Table 11. Of note, the three patients with PD that did not have P-SYN present on skin biopsy did not have evidence of REM sleep behavior disorder. Phosphorylated alpha-synuclein was deposited within sudomotor nerve fibers, pilomotor nerve fibers, vasomotor nerve fibers, nerve bundles and the sub-epidermal plexus.

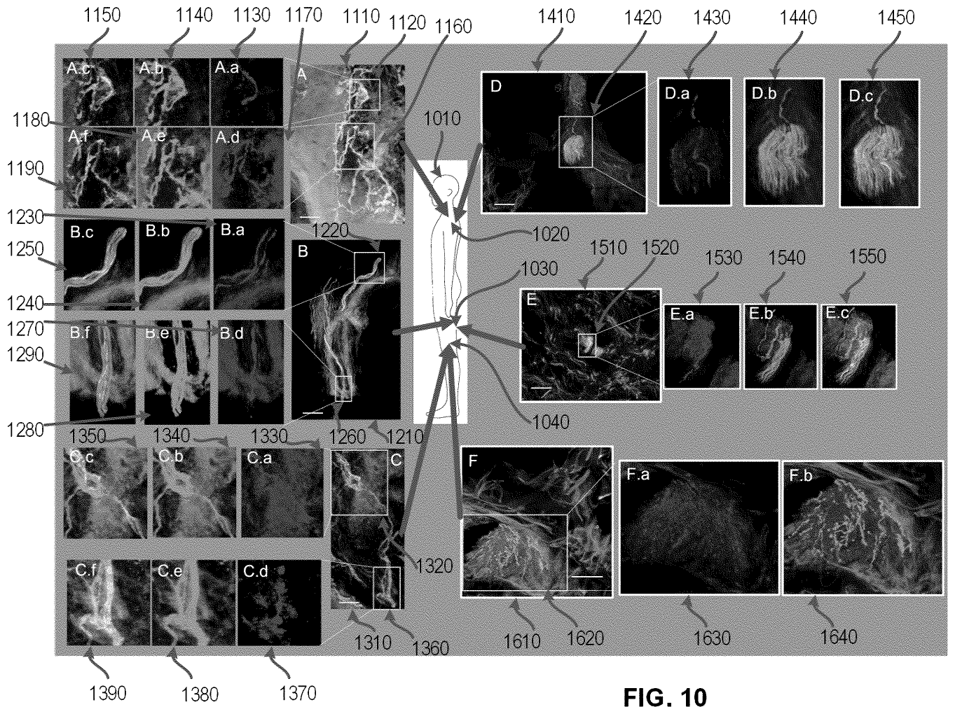

FIG. 10 illustrates the distribution of phosphorylated alpha-synuclein in PD and MSA. In FIG. 10, the 3 biopsy locations (distal leg 1040, distal thigh 1030, and posterior cervical region 1020) are shown on an exemplary patient 1010. Biopsies from a patient with MSA are shown in purple boxes (panels 1110, 1210, and 1310), the biopsy results from a patient with PD are shown in the pale green outlined boxes panels (1410, 1510, and 1610). As a reference, the white scale bar=50 micrometers for all panels in FIG. 10.

The biopsy from the posterior cervical region 1020 of a patient 1010 with MSA is shown in panel 1110, where multiple positive regions 1120, 1160 are seen in the sub-epidermal plexus. These are shown in more expanded detail in panels 1130, 1140, and 1150 for first positive region 1120 and in panels 1170, 1180, and 1190 for second positive region 1160, where phosphorylated alpha-synuclein is seen in red as shown in panels 1130 and 1170, the overlying nerve fibers stained with protein gene product 9.5 (PGP9.5) in green, as shown in panels 1140 and 1180, and the merged images, as shown in panels 1150 and 1190.

The biopsy from the distal thigh 1030 of a patient 1010 with MSA is shown in panel 1210, where multiple positive regions 1220, 1260 are seen in the sub-epidermal plexus. These are shown in more expanded detail in panels 1230, 1240, and 1250 for first positive region 1220 and in panels 1270, 1280, and 1290 for second positive region 1260, where phosphorylated alpha-synuclein is seen in red, as shown in panels 1230 and 1270, the overlying nerve fibers stained with protein gene product 9.5 (PGP9.5) in green, as shown in panels 1240 and 1280, and the merged images, as shown in panels 1250 and 1290.

The biopsy from the distal leg 1040 of a patient 1010 with MSA is shown in panel 1310, where multiple positive regions 1320, 1360 are seen in the sub-epidermal plexus. These are shown in more expanded detail in panels 1330, 1340, and 1350 for first positive region 1320 and in panels 1370, 1380, and 1390 for second positive region 1360, where phosphorylated alpha-synuclein is seen in red as shown in panels 1330 and 1370, the overlying nerve fibers stained with protein gene product 9.5 (PGP9.5) in green, as shown in panels 1340 and 1380, and the merged images, as shown in panels 1350 and 1390.

The biopsy from the posterior cervical region 1020 of a patient 1010 with PD is shown in panel 1410 where two adjacent positive fibers are seen. The yellow box 1420 expands the view of this with phosphorylated alpha-synuclein in red, as shown in panel 1430, the overlying nerve fibers stained with protein gene product 9.5 (PGP9.5) in green, as shown in panel 1440, and the merged image in as shown in panel 1450.

The biopsy from the distal thigh 1030 of a patient 1010 with PD is shown in panel 1510 where a single fiber containing phosphorylated alpha-synuclein is seen. The yellow box 1520 expands the view of this with phosphorylated alpha-synuclein in red, as shown in panel 1530, the overlying nerve fibers stained with protein gene product 9.5 (PGP9.5) in green, as shown in panel 1540, and the merged image in as shown in panel 1550.

The biopsy from the distal leg 1040 of a patient 1010 with PD is shown in panel 1610 where no single fiber containing phosphorylated alpha-synuclein is seen. The yellow box 1620 expands the view of this with no identified phosphorylated alpha-synuclein in red, as shown in panel 1630, the overlying nerve fibers stained with protein gene product 9.5 (PGP9.5) in green, as shown in panel 1640, and the merged image in as shown in panel 1650.

The biopsy from the distal leg 1040 of a patient 1010 with PD demonstrate regions where no phosphorylated alpha-synuclein is noted (panel 1630) although nerve fibers are clearly identified (panel 1640).

There was no phosphorylated alpha-synuclein detected within nociceptive sensory fibers (intraepidermal fibers). The quantitative analysis of phosphorylated alpha-synuclein revealed significantly higher levels in patients with MSA compared to PD (P<0.0001 for all biopsy sites; also see Table 10 and FIG. 10).

The topography of phosphorylated alpha-synuclein deposition differed between patients with MSA and PD. The deposition of phosphorylated alpha-synuclein in patients with PD followed a proximal-distal gradient, with the posterior cervical site positive in 93% of cases, the distal thigh positive in 33% of cases and the distal leg in 17% of cases. In contrast, deposition of alpha-synuclein in patients with MSA appeared more evenly distributed: the posterior cervical was positive in 84% of cases, the distal thigh in 87% of cases and the distal leg in 77% of cases. The topographic distribution was calculated using the distribution coefficient for P-SYN. In PD the distribution coefficient was 0.64±0.48 (indicating a posterior cervical predilection), while the distribution for MSA was 0.07±0.37 (indicating a more even distribution across all biopsy sites) (P<0.0001 PD vs MSA).

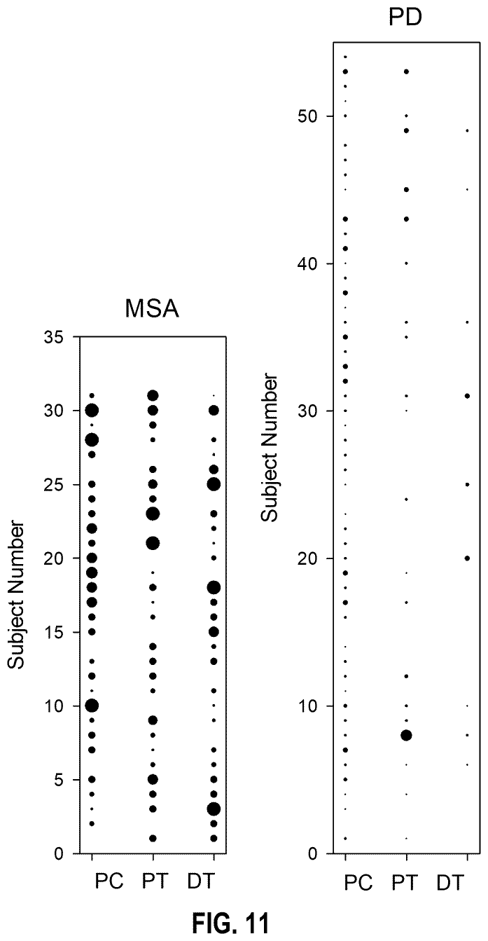

The topographical distribution and quantity of P-SYN deposition is illustrated in FIG. 11. The relative amount of synuclein deposition by biopsy site is shown for each individual subject. The larger the circle, the greater the synuclein deposition. The left panel of FIG. 11 is for subjects with MSA, the right panel of FIG. 11 is for individuals with PD. When comparing the two panels, P-SYN deposition is greater and more evenly distributed than in MSA than in PD. The key to FIG. 11 is PC: posterior cervical. DT: distal thigh. DL: distal leg.

In addition, the specific location of phosphorylated alpha-synuclein deposition within a given biopsy differed between patients with MSA and PD. In patients with MSA, P-SYN was detected in the subepidermal plexus region (composed of sensory afferent fibers) in 68% of cases (21/31) and only in 6% of PD patients (3/54).

There is a positive correlation in patients with MSA between total UMSARS and P-SYN deposition (R=0.632, P=0.001), however there was no correlation between the MDS-UPDRS and P-SYN deposition in patients with PD (R=0.2, P=0.14). In the combined MSA and PD groups there is a correlation between the Hohn and Yahr scores and P-SYN deposition (R=0.41, P<0.0001).

There are correlations in the combined MSA and PD groups with phosphorylated alpha-synuclein levels and autonomic dysfunction as measured by decreased heart rate variability to paced breathing (R=−0.29, P=0.008), reduced heart rate response to a Valsalva maneuver (R=−0.43, P<0.0001), the fall in blood pressure during phase 2 of the Valsalva maneuver (R=−0.26, P=0.017) and reduced blood pressure overshoot in response to a Valsalva maneuver (R=−0.58, P<0.0001). There were correlations between P-SYN deposition and supine systolic blood pressure (R=0.42, P<0.0001), supine diastolic blood pressure (R=0.43, P<0.0001), supine heart rate (R=0.58, P<0.0001 and fall in systolic blood pressure on upright tilt table test (R=−0.47, P<0.0001).

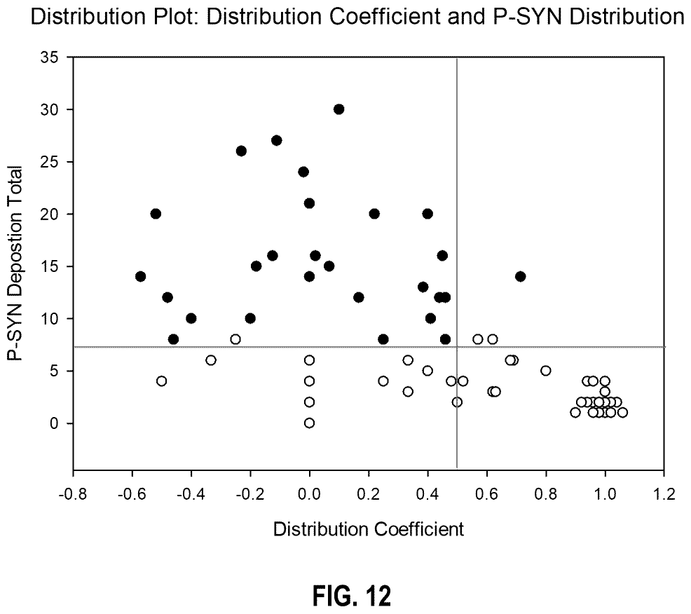

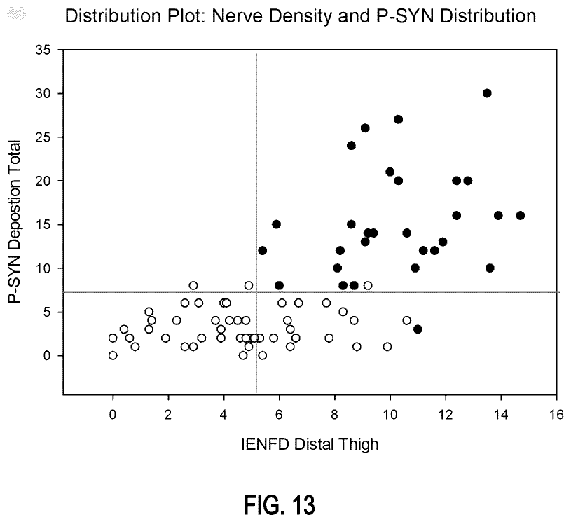

Sensitivity and specificity of any given measure to distinguish MSA from PD was calculated using ROC curves and the discrimination between groups (area under the ROC curve) is reported in Table 10 and FIGS. 12 and 13. FIG. 12 is a scatter plot of distribution coefficient and P-SYN distribution. FIG. 13 is a scatter plot of nerve density and P-SYN distribution. FIGS. 12 and 13 illustrate various test characteristics that distinguish MSA from PD. The plots of total phosphorylated alpha-synuclein are shown for patients with MSA (black circles) and PD (open circles). In FIG. 12, the plot of total phosphorylated against the synuclein distribution coefficient is shown. In FIG. 12, the cut-off thresholds identified using ROC curves are shown and are the values seen in the table confusion matrix in Table 12. In FIG. 12, the upper left quadrant indicates patients with a high probability of MSA. In FIG. 12, the cut-off thresholds identified using ROC curves are shown and are the values seen in the table confusion matrix in Table 12. In FIG. 13, the plot of total phosphorylated against the intra-epidermal nerve fiber density is shown. In FIG. 13, the upper right quadrant indicates patients with a high probability of MSA. In FIG. 12, the cut-off thresholds identified using ROC curves are shown and are the values seen in the table confusion matrix in Table 12. For both graphs, all other quadrants would indicate patients with PD. In both graphs, a single patient with MSA and PD are misclassified.

A synuclein signature that distinguished MSA from PD include: 1) The quantitation of P-SYN across all 3 biopsy sites (sensitivity of 96.8% and a specificity of 94.4% with a P-SYN threshold of ≥7.0; an AUC of 0.983); and 2) the distribution coefficient of P-SYN (sensitivity 77%, specificity 74% with a distribution coefficient threshold of <0.5 with an AUC of 0.826). Combining cut-off thresholds of P-SYN total≥7.0 and the distribution coefficient≤0.5 provides 96.8% sensitivity and 98.1% specificity at distinguishing MSA from PD. With reference to Table 11 and Table 12, the results are displayed in a confusion matrix format.

TABLE 11
Confusion matrix for P-SYN total in patients with MSA

| | P-SYN Total ≥7.0 | | |
|---|---|---|---|
| | | Positive | Negative |
| Diagnosis of MSA | Positive | 30 | 1 |
| | Negative | 3 | 51 |

Confusion matrix for total P-SYN with a cutoff threshold of ≥7.0 leads to a sensitivity of 96.8% and a specificity of 94.4% for the diagnosis of MSA.

TABLE 12
Confusion matrix for P-SYN total ≥7.0 AND a distribution
index of ≤0.5 in patients with MSA

| | P-SYN Total ≥7.0 AND | | |
|---|---|---|---|
| | distribution index ≤0.5 | | |
| | | Positive | Negative |
| Diagnosis of MSA | Positive | 30 | 1 |
| | Negative | 1 | 53 |

Confusion matrix using a combination of total P-SYN with a cutoff threshold of ≥7.0 combined with a distribution index of ≤0.5 leads to a sensitivity of 96.8% and a specificity of 98.1% for the diagnosis of MSA.

Other synuclein signatures of pathological combinations offered similar results. As illustrated in Table 13, the quantitation of P-SYN across all 3 biopsy sites (with a P-SYN threshold of 7.0) combined with the IENFD at the distal leg (with an IENFD threshold of 5.0 fibers/mm) provided a 96.8% sensitivity and a 98.1% specificity at distinguishing PD from MSA.

TABLE 13
Confusion matrix for P-SYN total ≥7.0 AND a IENFD
of ≤5.0 fibers/mm in patients with MSA

| | P-SYN Total ≥7.0 AND | | |
|---|---|---|---|
| | IENFD ≥5.0 fibers/mm | | |
| | | Positive | Negative |
| Diagnosis of MSA | Positive | 30 | 1 |
| | Negative | 1 | 53 |

Confusion matrix using a combination of total P-SYN with a cutoff threshold of ≥7.0 combined with a IENFD of ≥5.0 fibers/mm leads to a sensitivity of 96.8% and a specificity of 98.1% for the diagnosis of MSA.

The primary question in this exemplary study was to determine if skin biopsies with quantitation of phosphorylated alpha-synuclein and intra-epidermal nerve fiber density biopsy could differentiate MSA from PD. Patients with MSA and PD were recruited using standard clinical diagnostic criteria. The diagnostic test result and clinical status were defined by different individuals. This exemplary study provides Class II evidence that measurement of phosphorylated alpha-synuclein in skin biopsies can differentiate patients with MSA from those with PD.

This exemplary cohort study demonstrates that MSA patients can be differentiated from those with PD and healthy controls with excellent sensitivity and specificity using cutaneous neuropathological assessments combined with selected clinical features. Specifically, the study shows that: 1) phosphorylated alpha-synuclein is present within cutaneous autonomic nerve fibers in patients with MSA and PD, but not in healthy age-matched controls; 2) cutaneous phosphorylated alpha-synuclein deposition is greater in patients with MSA compared to patients with PD, 3) the topographic distribution of alpha-synuclein deposition differs in patients with MSA versus PD, namely, patients with PD showed a proximal to distal gradient, whereas deposition was more evenly distributed in patients with MSA; and, 4) Patients with PD have a mild length dependent sensory and autonomic neuropathy that is not present in patients with MSA. Collectively, these data suggest that skin biopsy may be an accurate method to pathologically confirm a diagnosis of a synucleinopathy and distinguish between MSA and PD.

As is consistent with other examples herein, the topographic distribution of P-SYN differs in PD compared to MSA. This difference was used to develop a distribution index that enhances the diagnostic discrimination of the test. The unique deposition pattern of phosphorylated alpha-synuclein seen in MSA provides a powerful tool to discriminate MSA from PD.

The results of this study provide support for the measurement of cutaneous alpha-synuclein deposition can be used as a prognostic biomarker in PD and MSA in clinical practice, research studies and clinical trials. The assessment of alpha-synuclein deposition within the skin can provide a safe, accessible, and repeatable marker of disease pathology.

This study determined that the presence of total alpha-synuclein and phosphorylated alpha-synuclein deposits in the autonomic nerve fibers from skin biopsies collected at three different sites (posterior cervical, distal thigh and distal leg) in a cohort consisting of 31 patients with MSA, 54 patients with PD, and 24 healthy controls (HC). The study determined that the presence of phosphorylated alpha-synuclein accumulation within cutaneous autonomic nerve fibers in both MSA (31/31) and PD (51/54) patients but not in HC (0/24). The deposition of phosphorylated alpha-synuclein is greater in patients with MSA and the topographic distribution of phosphorylated alpha-synuclein shows a proximal-distal gradient only in PD patients, while MSA patients show a more even distribution. The combination of levels and distribution coefficients of phosphorylated alpha-synuclein has a high sensitivity and specificity (>94%) for distinguishing MSA from PD. Combining either phosphorylated alpha-synuclein levels or distribution coefficients with heart rate in response to standing or smell test score allowed a complete separation between MSA and PD.

### Example 10

### Other Factors for Consideration in Determining Cutaneous α-Synuclein Signatures.

In addition, several potential other pathological synuclein signatures (such as reduced IENFD or PMNFD) could be included in a model with equally effective discriminatory power. Furthermore, the inclusion of other clinical data in the model (such as, for example, anosmia, UPSIT, orthostatic hypotension, heart rate response to orthostatic change, etc.) could be included in a model if specific clinical data has an effective discriminatory power. In addition, the model can include nerve fiber specific markers (such as tyrosine hydroxylase or dopamine beta-hydroxylase) to confirm the presence of P-SYN within sympathetic adrenergic nerve fibers.

The correlations between alpha-synuclein and clinical manifestations, e.g., physical examination scores, tests of autonomic function, autonomic symptoms and anosmia are understandably modest for both PD and MSA patients. Several factors may influence these relationships, and these have implications for the clinical use and interpretation of cutaneous alpha-synuclein measurement as a prognostic biomarker. Factors include that most patients were assessed and biopsied on symptomatic treatment which may have influenced the clinical assessments. Also, the relationship between central nervous system alpha-synuclein aggregation, central nervous system neurodegeneration and clinical manifestations is not linear, and if cutaneous alpha-synuclein deposition follows central deposition, similar non-linearity would be expected. Furthermore, discordance between the rate of disease progression in MSA and PD and alpha-synuclein deposition in the skin may influence the relationship.

The pathologic findings in Example 9 (decreased autonomic and small fiber sensory innervation in PD compared to MSA) are consistent with prior reports of peripheral nerve degeneration in Parkinson's disease and reduced orthostatic heart rate response. However, there is discordance between these findings and the observed amount and distribution of alpha-synuclein deposition. This observation highlights that the basis of these established phenotypic differences and the mechanism whereby alpha-synuclein causes neurodegeneration is not fully established. Possibilities to explain the phenotypic differences between the two disorders include: (1) differences at a cellular level, e.g., the recent identification of P-SYN in Schwann cells of patients with MSA but not the Lewy body diseases, and (2) differences at a molecular level, i.e., the different conformational changes in alpha-synuclein in the Lewy body disorders and MSA that have been observed in several studies.

The data in Example 9 does not provide a full explanation for the pathological and clinical phenotypic differences among the synucleinopathies. Example 9 is not designed to directly address this question, but does add to the information that surrounds the different distributions of central and peripheral nervous system alpha-synuclein deposition in MSA and PD.

The detection and differential distribution of alpha-synuclein in MSA and PD in Example 9 highlight the questions as to how and when alpha-synuclein is deposited in the cutaneous nerves and what is the topography of peripheral spread over time. Specifically, how does cutaneous alpha-synuclein fit into the Braak staging system, which hypothesizes that Lewy pathology starts in the olfactory bulb and enteric nervous system and invades the brain via the vagus nerve and possibly via a sympathetic spreading route that includes the sympathetic trunk and heart. A recent study has proposed the existence of 2 parkinsonian subtypes. One described as “bottom-up” or “body-first” and characterized by origin in the peripheral autonomic nervous system and ascent via the vagus and sympathetic autonomic nerves. This subtype is characterized by the occurrence of RBD prior to the onset of motor features. The second subtype, “topdown” or “brain first” is characterized by origin in the brain or olfactory tubercle with downward spread via the brainstem. In this subtype, RBD occurs after the appearance of motor features. Example 9 is not powered to assess the relationship of RBD to the amount and distribution of alpha-synuclein and the cross-sectional design does not allow for assessment of the changes in distribution and deposition.

Further understanding of the research and clinical utility of cutaneous alpha-synuclein measurement as a prognostic biomarker can be confirmed in a longitudinal study with clinical assessments, ideally off medications and carried out at multiple time points.

Cutaneous alpha-synuclein assessment can be used to measure target engagement of pharmacotherapy directed toward an alpha-synuclein target, and, in the long-term could be used as a surrogate biomarker, similar to the use of the amyloid PET scan in Alzheimer's disease and related disorders. As described herein, correlations are present with clinical manifestations of MSA and PD and phosphorylated alpha-synuclein levels and total alpha-synuclein ratios—the ratio normalizes deposition relative to nerve fiber density.

### Comparisons Between Example 1 and Example 9

In Example 1, if the P-SYN score is low (1-5) then i) With reduced IENFD at distal leg, or distal leg+distal thigh diagnosis is likely PD (85%). However, if evidence of phosphorylated alpha-synuclein within subepidermal plexus then diagnosis of PD is reduced to <30%, with MSA>70%.; and ii) Without reduced IENFD at any site, with age<65, and with phosphorylated alpha-synuclein deposition within subepidermal plexus diagnosis is likely MSA (>90%).

In Example 9, PD patients had a P-SYN of about 3.2 (see Table 11), which is within the low score of 1-5 of Example 1. In addition, the PD patents had a reduced IENFD at distal leg of about 4.6 and a reduced IENFD at distal leg+distal thigh of about 14 (lower than both the MSA patients and the control (P<0.01 leg and P<0.05 leg+thigh)), which gives a likely PD diagnosis of 85% according to Example 1. The results from Example 9 determined that patients with PD had reduced nerve fiber densities compared to patients with MSA (P<0.05, all fiber types), which is consistent with Example 1.

In Example 1, if the P-SYN score is medium (6-15) then ii) For age<70 without reduced IENFD, and phosphorylated alpha-synuclein present in sub-epidermal plexus MSA diagnosis>90%; and iii) With reduced IENFD at distal leg, or distal leg+distal thigh diagnosis is likely PD or DLB (>90%).

In Example 9, MSA patients had a P-SYN total of about 15 (see Table 11), which is within the medium score of 6-15 of Example 1, however, with the error bars of plus/minus 6.1, some of the MSA patients may be in the high score group of Example 1. In Example 9, the MSA patients did not have reduced IENFD at distal leg (P<0.01 compared to PD according to Table 11). According to Example 9, P-SYN was detected in the subepidermal plexus region (composed of sensory afferent fibers) in 68% of MSA patients (21/31) and only in 6% of PD patients (3/54). The detected P-SYN in the subepidermal plexus region without reduced IENFD of MSA patients in Example 9 is consistent with Example 1, which yields a likely MSA diagnosis of >90% for these patients.

In Example 1, if the P-SYN score is zero then diagnosis of synucleinopathy <5%. In Example 9, the control patients had a P-SYN total of 0. The results of Example 9 show phosphorylated alpha-synuclein is present within cutaneous autonomic nerve fibers in patients with MSA and PD, but not in healthy age-matched controls, which is consistent with Example 1.

A multi-faceted algorithm tool similar to the exemplary multi-faceted algorithm tool described in Example 1 can be used to transform the data collected in Example 9.

Various embodiments provide systems and methods for combining a patient's clinical features with a patient's deposition profile in accordance with Example 9; and calculating the probability the patient has MSA. The methods and systems can include accessing a data set consisting of a patient's clinical features, a patient's deposition profile, and a pathological signature of phosphorylated alpha-synuclein distribution; transforming a portion of the data set using a multi-faceted algorithm tool into a result that distinguishes a patient's diagnosis of MSA over PD.

In some embodiments, a multi-faceted algorithm tool for synucleinopathy fills a gap in scientific knowledge and provides a vertical advance in the field by (1) improving clinical accuracy (of synucleinopathy, and between synucleinopathy subtypes), (2) improving diagnostic discrimination in clinical trials, (3) accelerating the pace of research by providing an earlier diagnosis and (4) providing a novel surrogate endpoint of disease.

The multi-faceted algorithm tool would impact the burden of disease through improvements to clinical practice and by hastening the development of novel therapeutic treatments. In addition, the multi-faceted algorithm tool provides evidence of target engagement in clinical trials and accelerates progress towards disease modification.

Various embodiments provide a system for determining a treatment of a synucleinopathy disorder for a patient. The system can comprise: a multifaceted algorithm tool configured to: calculate a patient synuclein signature comprising a cutaneous alpha-synuclein distribution pattern, an assessment of intraepidermal sensory nerve fiber density and a dermal autonomic nerve fiber density; combine the patient synuclein signature with selected clinical information acquired from the patient; compare the patient synuclein signature and selected clinical information to a library of standard synuclein signatures and clinical information; and determine a patient's diagnosis of either MSA or PD

Various embodiments provide systems and methods for identifying the deposition of alpha-synuclein in a skin biopsy from a patient and can include determining if the patient has MSA. The methods and systems can include assessing a patient's deposition profile, as compared to a pathological signature of phosphorylated alpha-synuclein distribution for MSA; and calculating the probability the patient has MSA.

Various embodiments provide systems and methods for combining a patient's pathological features into a synuclein signature profile; and calculating the probability the patient has MSA. The synuclein signature profile can include determining the amount of phosphorylated alpha-synuclein in at least one skin biopsy, calculating the distribution of phosphorylated alpha-synuclein synuclein in at least one skin biopsy, and transforming the synuclein signature profile using a multi-faceted algorithm into a result that distinguishes a patient's diagnosis of MSA over PD. In some embodiments, the synuclein signature profile and patient clinic data can be entered into the multi-faceted algorithm, which transforms the profile and data result that distinguishes a patient's diagnosis of MSA or PD, with a probability of greater than 95%.

Various embodiments provide a synuclein signature for distinguishing MSA from PD in a patient, the synuclein signature comprises a quantitation of total P-SYN across a posterior cervical biopsy site, a distal thigh biopsy site, and a distal leg biopsy site, and a distribution coefficient of P-SYN across the posterior cervical biopsy site, the distal thigh biopsy site, and the distal leg biopsy site.

The system can comprise: a multifaceted algorithm tool configured to: calculate an intensity and a distribution of co-localized phosphorylated alpha-synuclein deposits within nerve fibers within a skin biopsy section of a patient; calculate an intra-epidermal nerve fiber density (IENFD) in the biopsy section of the patient; create a distribution score from the calculated distribution of co-localized phosphorylated alpha-synuclein deposits; create an intensity score from the calculated intensity of co-localized phosphorylated alpha-synuclein deposits; multiply the distribution score times the intensity score to determine a total score; enter clinical information is acquired from the patient; determine an patient outcome wherein: if the total score is 0, then a diagnosis of synucleinopathy<5%; if the total score is low, then diagnosis of PD, MSA, RBD or PAF phenoconverting to MSA>80%; if the total score is medium, then possible diagnosis of PD, MSA, RBD, PAF, or DLB; if the total score is higher, then likelihood of diagnosis of PD, DLB or PAF>90%; and if the total score is very high, then likelihood of diagnosis of DLB or PAF>90%.

Wherein the distribution score determines if no phosphorylated alpha-synuclein present, a single nerve fiber containing phosphorylated alpha-synuclein is detected, a few nerve fibers contain phosphorylated alpha-synuclein, but not the majority of tissue sections. nerve fibers containing phosphorylated alpha-synuclein are detected within at least many tissue sections, or nerve fibers containing phosphorylated alpha-synuclein are detected within every tissue section.

The method can comprise obtaining a skin biopsy sample from the patient; immunofluorescent staining the skin biopsy sample; imaging the skin biopsy sample; quantifying innervation of autonomic substructures in the skin biopsy sample; measuring phosphorylated α-synuclein within autonomic substructures in the skin biopsy sample; differentiating between the phosphorylated α-synuclein; inputting results from a clinical evaluation of the patient; and calculating a synucleinopathy treatment plan for the patient using the measured phosphorylated α-synuclein, as differentiated and the results from the clinical evaluation of the patient.

Various embodiments provide a system for determining a synucleinopathy disorder for a patient. The system can comprise a multifaceted algorithm tool configured to: calculate a patient synuclein signature comprising a cutaneous alpha-synuclein distribution pattern, an assessment of intraepidermal sensory nerve fiber density and a dermal autonomic nerve fiber density; combine the patient synuclein signature with selected clinical information acquired from the patient; compare the patient synuclein signature and selected clinical information to a library of standard synuclein signatures and clinical information; and determine a patient's diagnosis of either MSA or Parkinson's disease PD.

The patient synuclein signature is obtained from a biopsied skin sample from the patient. A reduced intraepidermal sensory nerve fiber density indicates the probability the patient has MSA is zero and the probability the patient has PD is at least 90%. The selected clinical information acquired from the patient can comprise at least one of heart rate in response to standing or smell test score.

Various embodiments provide a method for determining if the patient has MSA. The method can comprise calculating a deposition of alpha-synuclein in a skin biopsy from a patient; determining a cutaneous alpha-synuclein distribution pattern in the skin biopsy; determining a dermal autonomic nerve fiber density in the skin biopsy; determining a intraepidermal sensory nerve fiber density in the skin biopsy; calculating a patient synuclein signature comprising the cutaneous alpha-synuclein distribution pattern, the intraepidermal sensory nerve fiber density and the dermal autonomic nerve fiber density; assessing the patient synuclein signature as compared to a synuclein signature for MSA; and calculating the probability the patient has MSA.

The skin biopsy can be collected from three biopsy sites consisting of a posterior cervical biopsy site, a distal thigh biopsy site, and a distal leg biopsy site. The cutaneous alpha-synuclein distribution pattern can be a distribution coefficient (DC)=(PCscore-DLscore)/(PCscore+DTscore+DLscore), wherein the posterior cervical (PC), distal thigh (DT) and distal leg (DL) scores are the quantitative analysis of synuclein deposition at each biopsy site.

A score of zero in the numerator of the distribution coefficient are assigned a score of zero. A score of (+)1 indicates P-SYN is located only at the posterior cervical biopsy site. A score of (−)1 indicates P-SYN is located at only the distal leg biopsy site. A score between (+)1 and (−)1 indicates the relative distribution of P-SYN is a gradient from the posterior cervical biopsy site to the distal leg biopsy site.

If phosphorylated alpha-synuclein is not present within cutaneous autonomic nerve fibers, then the probability the patient has MSA is zero. A reduced intraepidermal sensory nerve fiber density indicates the probability the patient has MSA is zero and the probability the patient has PD is at least 90%.

Various embodiments provide a synuclein signature for distinguishing MSA from (PD in a patient. The synuclein signature can comprise a quantitation of total P-SYN across a posterior cervical biopsy site, a distal thigh biopsy site, and a distal leg biopsy site; and a distribution coefficient of P-SYN across the posterior cervical biopsy site, the distal thigh biopsy site, and the distal leg biopsy site wherein cut-off thresholds of total P-SYN≥7.0 and the distribution coefficient≤0.5 provides at least 95% sensitivity and at least 95% specificity at distinguishing MSA from PD.

The distribution coefficient (DC)=(PCscore−DLscore)/(PCscore+DTscore+DLscore), wherein the posterior cervical (PC), distal thigh (DT) and distal leg (DL) scores are the quantitative analysis of synuclein deposition at each biopsy site. If the quantitation of total P-SYN is zero, then the patient has neither MSA or PD.

Various embodiments provide a method for generating a synucleinopathy treatment plan for a patient. The method can comprise obtaining a skin biopsy sample from the patient; immunofluorescent staining the skin biopsy sample; imaging the skin biopsy sample; quantifying innervation of autonomic substructures in the skin biopsy sample; measuring phosphorylated α-synuclein within autonomic substructures in the skin biopsy sample; differentiating between the phosphorylated α-synuclein; inputting results from a clinical evaluation of the patient; and calculating a synucleinopathy treatment plan for the patient using the measured phosphorylated α-synuclein, as differentiated and the results from the clinical evaluation of the patient.

In the foregoing description, the present invention has been described with reference to specific exemplary embodiments. The particular implementations shown and described are illustrative of the present invention and its best mode and are not intended to otherwise limit the scope of the present invention in any way. Indeed, for the sake of brevity, conventional manufacturing, connection, preparation, and other functional aspects of the method and system may not be described in detail. Furthermore, the connecting lines shown in the various figures are intended to represent exemplary functional relationships and/or steps between the various elements. Many alternative or additional functional relationships or physical connections may be present in a practical system.

It should be understood that steps within a method may be executed in different order without altering the principles of the present invention. For example, various embodiments may be described herein in terms of various functional components and processing steps. It should be appreciated that such components and steps may be realized by any number of hardware components configured to perform the specified functions. Additionally, the components and/or elements recited in any embodiment may be assembled or otherwise operationally configured in a variety of permutations to produce substantially the same result as the present invention and are accordingly not limited to the specific configuration recited in the specific examples.

As used herein, the phrase “at least one of A, B, and C” can be construed to mean a logical (A or B or C), using a non-exclusive logical “or,” however, can be contrasted to mean (A, B, and C), in addition, can be construed to mean (A and B) or (A and C) or (B and C). As used herein, the phrase “A, B and/or C” should be construed to mean (A, B, and C) or alternatively (A or B or C), using a non-exclusive logical “or.”

The present invention has been described above with reference to various exemplary embodiments and examples, which are not intended to be limiting in describing the full scope of systems and methods of this invention. However, those skilled in the art will recognize that equivalent changes, modifications and variations of the embodiments, materials, systems, and methods may be made within the scope of the present invention, with substantially similar results, and are intended to be included within the scope of the present invention, as set forth in the following claims.

1. A system for determining a synucleinopathy disorder for a patient, the system comprising:
a multifaceted algorithm tool configured to:
calculate a patient synuclein signature comprising a cutaneous alpha-synuclein distribution pattern, an assessment of intraepidermal sensory nerve fiber density and a dermal autonomic nerve fiber density;
combine the patient synuclein signature with selected clinical information acquired from the patient;
compare the patient synuclein signature and selected clinical information to a library of standard synuclein signatures and clinical information; and
determine a patient's diagnosis of either multiple system atrophy (MSA) or Parkinson's disease (PD).

2. The system according to claim 1, wherein the patient synuclein signature is obtained from a biopsied skin sample from the patient.

3. The system according to claim 1, wherein a reduced intraepidermal sensory nerve fiber density indicates the probability the patient has MSA is zero and the probability the patient has Parkinson's disease (PD) is at least 90%.

4. The system according to claim 1, selected clinical information acquired from the patient comprises at least one of heart rate in response to standing or smell test score.

5. A method for determining if the patient has multiple system atrophy (MSA), the method comprising:
calculating a deposition of alpha-synuclein in a skin biopsy from a patient;
determining a cutaneous alpha-synuclein distribution pattern in the skin biopsy;
determining a dermal autonomic nerve fiber density in the skin biopsy;
determining a intraepidermal sensory nerve fiber density in the skin biopsy;
calculating a patient synuclein signature comprising the cutaneous alpha-synuclein distribution pattern, the intraepidermal sensory nerve fiber density and the dermal autonomic nerve fiber density;
assessing the patient synuclein signature as compared to a synuclein signature for MSA; and
calculating the probability the patient has MSA.

6. The method according to claim 5, wherein the skin biopsy is collected from three biopsy sites consisting of a posterior cervical biopsy site, a distal thigh biopsy site, and a distal leg biopsy site.

7. The method according to claim 6, wherein the cutaneous alpha-synuclein distribution pattern comprises a distribution coefficient (DC)=(PCscore−DLscore)/(PCscore+DTscore+DLscore), wherein the posterior cervical (PC), distal thigh (DT) and distal leg (DL) scores are the quantitative analysis of synuclein deposition at each biopsy site.

8. The method according to claim 7, wherein a score of zero in the numerator are assigned a score of zero.

9. The method according to claim 7, wherein a score of (+)1 indicates P-SYN is located only at the posterior cervical biopsy site.

10. The method according to claim 7, wherein a score of (−)1 indicates P-SYN is located at only the distal leg biopsy site.

11. The method according to claim 7, wherein a score between (+)1 and (−)1 indicates the relative distribution of P-SYN is a gradient from the posterior cervical biopsy site to the distal leg biopsy site.

12. The method according to claim 5, wherein if phosphorylated alpha-synuclein is not present within cutaneous autonomic nerve fibers, then the probability the patient has MSA is zero.

13. The method according to claim 5, wherein a reduced intraepidermal sensory nerve fiber density indicates the probability the patient has MSA is zero and the probability the patient has Parkinson's disease (PD) is at least 90%.

14. A synuclein signature for distinguishing multiple system atrophy (MSA) from Parkinson's disease (PD) in a patient, the synuclein signature comprising:
a quantitation of total P-SYN across a posterior cervical biopsy site, a distal thigh biopsy site, and a distal leg biopsy site; and
a distribution coefficient of P-SYN across the posterior cervical biopsy site, the distal thigh biopsy site, and the distal leg biopsy site
wherein cut-off thresholds of total P-SYN≥7.0 and the distribution coefficient ≤0.5 provides at least 95% sensitivity and at least 95% specificity at distinguishing MSA from PD.

15. The synuclein signature distribution according to claim 14, wherein the distribution coefficient (DC)=(PCscore−DLscore)/(PCscore+DTscore+DLscore), wherein the posterior cervical (PC), distal thigh (DT) and distal leg (DL) scores are the quantitative analysis of synuclein deposition at each biopsy site.

16. The synuclein signature distribution according to claim 14, wherein if the quantitation of total P-SYN is zero, then the patient has neither MSA or PD.

17. A method for generating a synucleinopathy treatment plan for a patient, the method comprising:
obtaining a skin biopsy sample from the patient;
immunofluorescent staining the skin biopsy sample;
imaging the skin biopsy sample;
quantifying innervation of autonomic substructures in the skin biopsy sample;
measuring phosphorylated α-synuclein within autonomic substructures in the skin biopsy sample;
differentiating between the phosphorylated α-synuclein;
inputting results from a clinical evaluation of the patient; and
calculating a synucleinopathy treatment plan for the patient using the measured phosphorylated α-synuclein, as differentiated and the results from the clinical evaluation of the patient.
